(19) United States
(12) Patent Application Publication
(10) Pub. No.: US 2026/0049315 A1
(43) Pub. Date: Feb. 19, 2026

(54) SOD1 DUAL EXPRESSION VECTORS AND USES THEREOF

(71) Applicant: University of Massachusetts, Westborough, MA (US)

(72) Inventors: Christian Mueller, Worcester, MA (US); Robert H. Brown, JR., Worcester, MA (US)

(73) Assignee: University of Massachusetts, Westborough, MA (US)

(21) Appl. No.: 19/171,594

(22) Filed: Apr. 7, 2025

Related U.S. Application Data
(63) Continuation of application No. 18/344,926, filed on Jun. 30, 2023, now Pat. No. 12,312,587.
Application No. 18/344,926 is a division of application No. 16/649,164, now Pat. No. 11,739,330.
(60) Provisional application No. 62/561,932, filed on Sep. 22, 2017.

(51) Int. Cl. C12N 15/113; A61K 35/76; A61K 48/00; C12N 7/00; C12N 15/86

(52) U.S. Cl. CPC C12N 15/1137; A61K 35/76; C12N 7/00; C12N 15/86; A61K 48/00; C12N 2750/14043

(57) ABSTRACT

In some aspects, the disclosure relates to compositions and methods useful for inhibiting SOD1 expression in cells (e.g., cells of a subject). In some embodiments, the disclosure describes isolated nucleic acids engineered to express an inhibitory nucleic acid targeting endogenous SOD1 and an mRNA encoding a hardened SOD1 protein. In some embodiments, compositions and methods described by the disclosure are useful for treating Amyotrophic Lateral Sclerosis (ALS) in a subject.

### RELATED APPLICATIONS

This application claims the benefit under 35 U.S.C. 119(e) of the filing date of U.S. Provisional Application Ser. No. 62/561,932, filed Sep. 22, 2017, entitled “SOD1 DUAL EXPRESSION VECTORS AND USES THEREOF”, the entire contents of which are incorporated herein by reference.

### BACKGROUND

Amyotrophic lateral sclerosis (ALS) is a progressive, generally fatal motor neuron disorder that sometimes develops concurrently with frontotemporal dementia (FTD). ALS is encountered in both sporadic (SALS) and familial (FALS) forms. About 10% % of cases are transmitted as autosomal dominant traits. An FDA-approved therapy for ALS is riluzole, a compound that prolongs survival by about 10%.

Generally, studies showing benefit of SOD1 silencing in ALS cells and transgenic animals have not described silencing only the mutant allele. Rather, in most studies the silencing reduces levels of both the mutant, toxic SOD1 protein and also the wildtype SOD1 protein. However, excessive silencing of SOD1 from both the mutant and the wild-type alleles might relate to undesirable biological consequences as a result of reducing activity or function of wild-type SOD1 protein.

### SUMMARY

Aspects of the disclosure relate to compositions and methods for modulating cytosolic Cu/Za superoxide dismutase (SOD1) expression in cells. Accordingly, in some embodiments, methods are provided that are useful for treating ALS. In some embodiments, the disclosure provides synthetic nucleic acids (e.g., a synthetic microRNA) engineered to inhibit expression of endogenous SOD1 in cells or a subject. In some embodiments, the disclosure provides a nucleic acid engineered to express exogenous SOD1 in cells or a subject. In some embodiments, such exogenous SOD1 is resistant to targeting by a synthetic nucleic acid (e.g., a synthetic microRNA) that targets endogenous SOD1. Accordingly, in some embodiments, the disclosure provides compositions and methods for coupling the delivery of (1) a synthetic microRNA to silence expression of endogenous cytosolic Cu/Za superoxide dismutase (SOD1) activity, with (2) a second construct to express exogenous SOD1 resistant to the synthetic microRNA (miRNA).

The disclosure is based, in part, on compositions and methods described here that address the challenge of loss of neuroprotective activity from SOD1 dismotation by including in series with an anti-SOD1 miRNA, a cDNA for SOD1 expressed from an RNA engineered to be resistant to the anti-SOD1 miRNA. In some embodiments, constructs described by the disclosure, allow for normal levels of SOD1 dismutation activity (e.g., in a cell or subject that has been administered the construct) even with total silencing of both WT and mutant endogenous SOD1 alleles.

Accordingly, in some aspects, the disclosure provides an isolated nucleic acid comprising: a first region that encodes one or more first miRNAs comprising a nucleic acid having sufficient sequence complementary with an endogenous mRNA of a subject to hybridize with and inhibit expression of the endogenous mRNA, wherein the endogenous mRNA encodes a SOD1 protein; and a second region encoding an exogenous mRNA that encodes a wild-type SOD1 protein, wherein the one or more first miRNAs do not comprise a nucleic acid having sufficient sequence complementary to hybridize with and inhibit expression of the exogenous mRNA.

In some embodiments, an exogenous mRNA lacks a 5′ untranslated region (5′ UTR), lacks a 3° untranslated region (3′ UTR), or lacks both a 5′ UTR and a 3′UTR.

In some embodiments, an exogenous mRNA encoding the SOD1 protein has one or more silent base pair mutations relative to the endogenous mRNA. In some embodiments, an exogenous mRNA comprises a nucleic acid sequence that is at least 95% identical to the endogenous miRNA.

In some embodiments, the wild-type SOD1 is encoded by a nucleic acid sequence set forth in SEQ ID NO: 7 (Hardened SOD1 sequence).

In some embodiments, one or more first miRNAs targets an untranslated region (e.g. 5 UTR or 3′UTR) of a nucleic acid encoding an endogenous mRNA. In some embodiments, one or more first miRNAs targets a coding sequence of a nucleic acid encoding an endogenous mRNA.

In some embodiments, one or more first miRNAs hybridizes to a nucleic acid comprising 5.6, 7, 8, 9, 10, 11, 12, 13, 14, 15, 16, 17, 18, 19, 20 or 21 consecutive nucleotides of a RNA encoded by the sequence as set forth in SEQ ID NO: 3. In some embodiments, one or more first miRNAs hybridizes to a nucleic acid comprising 5, 6, 7, 8, 9, 10, 11, 12, 13, 14, 15, 16, 17, 18, 19, 20 or 21 consecutive nucleotides of a RNA encoded by the sequence as set forth in SEQ ID NO: 2.

In some embodiments, one or more first miRNAs comprises or is encoded by 5, 6, 7, 8, 9, 10, 11, 12, 13, 14, 15, 16, 17, 18, 19, 20 or 21 consecutive nucleotides of a sequence as set forth in SEQ ID NO: 4. In some embodiments, one or more first miRNAs comprises or is encoded by 5, 6, 7, 8, 9, 10, 11, 12, 13, 14, 15, 16, 17, 18, 19, 20 or 21 consecutive nucleotides of a sequence as set forth in SEQ ID NO: 3. In some embodiments, an miRNA further comprises flanking regions of miR-155 or flanking regions of miR-30.

In some embodiments, an isolated nucleic acid further comprises a first promoter. In some embodiments, a first promoter is operably linked to a first region of an isolated nucleic acid as described by the disclosure.

In some embodiments, a first promoter is a RNA polymerase III (pol III) promoter, such as an H1 promoter or a U6 promoter.

In some embodiments, a first promoter is a RNA polymerase II (pol II) promoter, such as a chicken beta actin (CBA) promoter, or an endogenous SOD1 promoter (e.g., SEQ ID NO: 16). In some embodiments, an isolated nucleic acid further comprises a second promoter. In some embodiments, a second promoter is operably linked to a second region of an isolated nucleic acid as described by the disclosure.

In some embodiments, a second promoter is a pol II promoter, such as a chicken beta actin (CBA) promoter, or an endogenous SOD1 promoter.

In some embodiments, an isolated nucleic acid further comprises an enhancer sequence, such as a cytomegalovirus (CMV) enhancer.

In some embodiments, a first region is positioned within an untranslated region (e.g., UTR) of a second region. In some embodiments, a first region is positioned within an intron of an isolated nucleic acid. In some embodiments, a first region is positioned 5′ with respect to a second region.

In some embodiments, an isolated nucleic acid further comprises at least one adeno-associated virus (AAV) inverted terminal repeat (ITR). In some embodiments, an isolated nucleic acid comprises a full-length ITR and a mutant ITR. In some embodiments, ITRs flank the first and second regions of an isolated nucleic acid as described by the disclosure.

In some embodiments, the disclosure provides a recombinant adeno-associated virus (rAAV) comprising an isolated nucleic acid as described by the disclosure and an AAV capsid protein.

In some embodiments, a rAAV targets CNS tissue. In some embodiments, a rAAV targets neurons.

In some embodiments, a capsid protein is AAV9 capsid protein or AAVrh.10 capsid protein.

In some aspects, the disclosure provides a composition comprising an isolated nucleic as described by the disclosure, or an rAAV as described by the disclosure, and a pharmaceutically acceptable excipient.

In some aspects, the disclosure provides a method for inhibiting SOD1 expression in a cell, the method comprising delivering to a cell an isolated nucleic acid as described by the disclosure, or an rAAV as described by the disclosure.

In some embodiments, a cell comprises a nucleic acid sequence encoding a mutant SOD1 protein.

In some aspects, the disclosure provides a method for treating a subject having or suspected of having ALS, the method comprising administering to the subject an effective amount of an isolated nucleic acid as described by the disclosure, or an effective amount of an rAAV as described by the disclosure.

In some embodiments, a subject comprises a nucleic acid sequence encoding a mutant SOD1 protein. In some embodiments, a subject is a mammalian subject, such as a human subject.

### BRIEF DESCRIPTION OF DRAWINGS

FIG. 1 shows a schematic overview of construct design for a bicistronic dual function vector. The anti-Sod1 miRNA is expressed by an H1 promoter and the miRNA-resistant SOD1 cDNA is expressed by a chicken beta actin promoter and CMV enhancer (e.g., CAG promoter).

FIG. 2 shows a schematic overview of construct design for a single promoter dual function vector. The anti-Sod1 miRNA and miRNA-resistant SOD1 eDNA are both expressed by a chicken beta actin promoter and CMV enhancer (e.g., CAG promoter). The anti-Sod1 miR is located in an intron.

FIG. 3 shows a schematic overview of construct design for a bicistronic dual function vector. The anti-Sod1 miRNA is expressed by an H1 promoter and the miRNA-resistant SOD1 cDNA is expressed by a chicken beta actin promoter and CMV enhancer (e.g., CAG promoter). The focus of the SOD1 cDNA containing a silent mutation relative to wild-type SOD1 is shown (“miR-SOD Resistant Target”).

FIG. 4 shows a schematic overview of construct design for a single promoter dual function vector. The anti-Sod1 miRNA and miRNA-resistant SOD1 cDNA are both expressed by a chicken beta actin promoter and CMV enhancer (e.g., CAG promoter). The locus of the SOD1 cDNA containing a silent mutation relative to wild-type SOD1 is shown (“miR-SOD Resistant Target”). The anti-Sod1 miR is located in an intron.

FIG. 5 shows a schematic overview of construct design for a bicistronic dual function self-complementary AAV vector. The anti-Sod1 miRNA is expressed by an H1 promoter and the miRNA-resistant SOD1 cDNA is expressed by a chicken beta actin promoter and CMV enhancer (e.g., CAG promoter). The locus of the SOD1 cDNA containing a silent mutation relative to wild-type SOD1 is shown (“miR-SOD Resistant Target”). A mutant AAV inverted terminal repeat (ITR) is present on the 5′ end of the construct and a full-length AAV ITR is located at the 3′ end.

FIG. 6 shows a schematic overview of construct design for a bicistronic dual function self-complementary AAV vector. The anti-Sod1 miRNA is expressed by an H1 promoter and the miRNA-resistant SOD1 cDNA is expressed by a chicken beta actin promoter and CMV enhancer (e.g., CAC promoter). The locus of the SOD1 cDNA containing a silent mutation relative to wild type SOD1 is shown (“miR-SOD Resistant Target”). The SOD1 expression construct lacks a 3′UTR. A mutant AAV inverted terminal repeat (ITR) is present on the 5′ end of the constrict and a full-length AAV ITR is located at the 3′ end.

FIG. 7 shows a schematic overview of construct design for a single promoter dual function AAV vector. The anti-Sod1 miRNA and miRNA-resistant SOD1 cDNA are both expressed by a chicken beta actin promoter and CMV enhancer (e.g., CAG promoter). The locus of the SOD1 cDNA containing a silent mutation relative to wild-type SOD1 is shown (“miR-SOD Resistant Target”). The anti-Sod1 miR is located in an intron. AAV ITRs are located at the 5′ and 3′ ends of the construct.

FIG. 8 shows a schematic overview of construct design for a single promoter dual function AAV vector. The anti-Sod1 miRNA and miRNA-resistant SOD1 cDNA are both expressed by a chicken beta actin promoter and CMV enhancer (e.g., CAG promoter). The locus of the SOD1 cDNA containing a silent mutation relative to wild-type SOD1 is shown (“miR-SOD Resistant Target”). The SOD1 expression construct lacks a J′UTR. The anti-Sod1 miR is located in an intron. AAV ITRs are located at the S′ and 3′ ends of the construct.

FIG. 9 shows a nucleic acid sequence alignment of wild-type SOD1 coding sequence (SEQ ID NO: 1) with an example of a “hardened” SOD1 coding sequence (SEQ ID NO: 7).

### DETAILED DESCRIPTION

In some aspects, the disclosure relates to compositions and methods for modulating expression and/or activity of genes associated with amyotrophic lateral sclerosis (ALS) in cells (e.g., cells of a subject). For example, in some aspects, the disclosure provides compositions (e.g., dual function vectors) that simultaneously express in cells or a subject (i) one or more synthetic nucleic acids (e.g., inhibitory RNAs, such as miRNAs, shRNAs, etc.) that inhibits a gene associated with ALS and (ii) an exogenous gene associated with ALS that encodes a protein that is resistant to the synthetic nucleic acid. Examples of genes associated with ALS include but are not limited to C9Orf72, SOD1, FUS, TARDBP, SQSTM1, VCP, OPTN, PFN1, UBQLN2, DCTN1, ALS2, CHMP2B, FIG4, HNRNAP1, ATXN2, ANG, SPG11, APB, NEFH, CHCHD10, ERBB4, PRPH, MATR3, SETX, SIGMAR1, TBK1, TRPM7, TUBA4A, ANXA11, NEK1, SARM1, UN13A, MOBP, SCFD1, C21Orf2, and others described, for example by Renton et al. (2014) Nature Neuroscience 17 (1): 17-23. In some embodiments, the gene associated with ALS is a dominant negative gene associated with ALS (e.g., a gene encoding a dominant negative gene product, such as a protein, that is associated with ALS).

Aspects of the disclosure relate to compositions and methods for modulating cytosolic Cu/Zn superoxide dismutase (SOD1) expression in cells. Accordingly, in some embodiments, methods are provided that are useful for treating ALS. In some embodiments, the disclosure provides synthetic nucleic acids (e.g., a synthetic microRNA) engineered to inhibit expression of endogenous SOD1 in cells or a subject. In some embodiments, the disclosure provides a nucleic acid engineered to express exogenous SOD1 in cells or a subject. In some embodiments, such exogenous SOD1 is resistant to targeting by a synthetic nucleic acid (e.g., a synthetic microRNA) that targets endogenous SOD1.

Aspects of the disclosure relate to improved gene therapy compositions and related methods for treating ALS using the recombinant adeno-associated viral (rAAV) vectors. In particular, rAAVs are provided that harbor nucleic acids engineered to express inhibitory nucleic acids that silence genes, such as SOD1, which are associated with ALS. In some embodiments, the disclosure utilizes a recombinant AAV (e.g., rAAV9, rAAV.Rh10, etc.) to deliver a microRNA to the CNS and thereby silence an ALS gene, such as SOD1. In some aspects, the disclosure relates to the discovery of dual function vectors that are capable of knocking-down endogenous SOD1 expression (e.g., wild-type SOD1 and mutant SOD1 expression) in a subject while expressing wild type SOD1. Accordingly, constructs described by the disclosure, in some embodiments, allow for normal levels of SOD1 dismutation activity (e.g., in a cell or subject that has been administered the construct) even with total silencing of both WT and mutant endogenous SOD1 alleles.

In some aspects, the disclosure provides an isolated nucleic acid comprising: a first region that encodes one or more first miRNAs comprising a nucleic acid having sufficient sequence complementary with an endogenous mRNA of a subject to hybridize with and inhibit expression of the endogenous mRNA, wherein the endogenous mRNA encodes a SOD1 protein; and a second region encoding an exogenous mRNA that encodes a wild-type SOD1 protein, wherein the one or more first miRNAs do not comprise a nucleic acid having sufficient sequence complementary to hybridize with and inhibit expression of the exogenous mRNA.

### SOD1

As used herein, “SOD1” refers to Superoxide dismutase (SOD1), which is an enzyme encoded in humans by the SOD1 gene. Typically. SOD1 functions to catalyze disproportionation of superoxide to hydrogen peroxide and dioxygen, and remove free radicals in the body, “Wild-type SOD1” refers to a gene product (e.g., protein) encoded by a SOD1 gene that does not cause gain of function toxicity in a cell or subject (e.g., that does not or will not result in the development of ALS). In some embodiments, a wild-type SOD1 gene encodes an mRNA transcript (e.g., a mature mRNA transcript) having a sequence set forth in NCBI Accession No. NM_000454.4.

“Mutant SOD1” refers to a gene product (e.g., protein) comprising one or more mutations (e.g., missense mutations, nonsense mutations, frameshift mutations, insertions, deletions, etc.) that result in the gene product (e.g., protein) having an altered function, such as a toxic gain of function. Generally, a nucleic acid encoding a mutant SOD1 gene product does not comprise any silent mutations relative to a nucleic acid encoding a wild-type SOD1 gene product.

Mutations in the gene encoding Superoxide dismutase (SOD1), located on chromosome 21, have been linked to familial amyotrophic lateral sclerosis. Superoxide dismutase (SOD1) is an enzyme encoded by the SOD1 gene. SOD1 hinds copper and zine ions and is one of three superoxide dismutases responsible for destroying free superoxide radicals in the body. The encoded isozyme is a soluble cytoplasmic and mitochondrial intermembrane space protein, acting as a homodimer to convert naturally occurring, but harmful superoxide radicals to molecular oxygen and hydrogen peroxide. Frequent SOD1 mutations that occur and cause ALS include A4V, H46R and G93A. Additional SOD1 mutations are described, for example by Banci et al. (2008) PLOS ONE 3 (2): e1677.

The disclosure is based, in part, on the discovery that nucleic acid constructs that simultaneously inhibit endogenous SOD1 expression in a non-allele-specific manner (e.g. silence endogenous wild-type and endogenous mutant SOD1) and express an exogenous SOD1 protein (e.g., express an exogenous wild-type SOD1 or an exogenous hardened SOD1 protein) allow for normal levels of SOD1 dismutation activity even with total silencing of both WT and mutant endogenous SOD1 alleles. As used herein, “endogenous” refers to a gene (e.g., a SOD1 gene) or a gene product (e.g., a SOD1 protein) that is encoded by the native DNA of a cell. “Exogenous” refers to a gene (e.g., a nucleic acid encoding a SOD1 protein, such as SOD1 cDNA) or a gene product (e.g. a SOD1 protein, such as a hardened SOD1 protein) that originates from a source other than the native DNA of a cell (e.g., has been introduced to a cell non-naturally).

In some embodiments, an exogenous SOD1 nucleic acid sequence encodes a hardened SOD1 protein. As used herein. “hardened SOD1” refers to a nucleic acid sequence encoding a SOD1 protein that comprises one or more silent mutations such that it encodes the same protein as an endogenous wild-type SOD1 protein but has a different primary nucleic acid (e.g., DNA) sequence. Without wishing to be bound by any particular theory, a “hardened SOD1” mRNA transcript is not inhibited by certain inhibitory RNAs (e.g., miRNAs) that target endogenous SOD1 RNA transcripts (e.g., wild-type SOD1 and mutant SOD1 transcripts).

The number of silent mutations in a hardened SOD1 nucleic acid sequence can vary. In some embodiments, a nucleic acid sequence encoding a hardened SOD1 comprises between about 1 and about 50 (e.g., any integer between 1 and 50, inclusive) silent mutations relative to a wild-type SOD1 nucleic acid sequence (e.g., SEQ ID NO: 1; SOD1 coding sequence). In some embodiments, a nucleic acid sequence encoding a hardened SOD1 comprises at least 1, at least 2, at least 3, at least 4, at least 5, at least 6, at least 7, at least 8, at least 9, at least 10), at least 11, at least 12, at least 13, at least 14, or at least 15 silent mutations relative to a wild-type SOD1 nucleic acid sequence (e.g., SEQ ID NO: 1; SOD1 coding sequence). In some embodiments, one or more silent mutations of a nucleic acid sequence encoding a hardened SOD1 are located in a seed region targeted by an inhibitory nucleic acid. In some embodiments, a seed region ranges from about 3 to about 25 continuous nucleotides in length (e.g., any integer between 3 and 25, inclusive).

The nucleic acid (e.g., DNA) sequence identity between a nucleic acid encoding an exogenous (e.g., hardened) SOD1 protein and an endogenous wild-type SOD1 protein can vary. In some embodiments, a nucleic acid sequence encoding an exogenous SOD1 protein is between about 99.9% and about 85% identical to an endogenous wild-type SOD1 nucleic acid sequence (e.g., SEQ ID NO: 1; SOD1 DNA coding sequence). In some embodiments, a nucleic acid sequence encoding an exogenous SOD1 protein is about 99.99%, about 99%, about 98%, about 97%, about 96%, about 959%, about 94%, about 939%, about 92%, about 91%, about 90%, about 898, about 88%, about 875%, about 86%, or about 85% identical to an endogenous wild-type SOD1 nucleic acid sequence (e.g., SEQ ID NO: 1; SOD1 DNA coding sequence). In some embodiments, a nucleic acid sequence encodes an exogenous SOD1 protein having an amino acid sequence that is between about 99.9% and about 90% (e.g. bout 99.96%, about 99%, about 98%, about 97%, about 96%, about 95%, about 94%, about 93%, about 92%, about 91%, or about 90%) identical to an endogenous wild type SOD1 amino acid sequence (e.g., SEQ ID NO: 17).

### Inhibitory Nucleic Acids

Aspects of the disclosure relate to inhibitory nucleic acids targeting SOD1 (e.g., endogenous SOD1). In some embodiments, the inhibitory nucleic acid is a nucleic acid that hybridizes to at least a portion of the target nucleic acid, such as an RNA, pre-mRNA, mRNA, and inhibits its function or expression. In some embodiments, the inhibitory nucleic acid is single stranded or double stranded. In some embodiments, the inhibitory nucleic acid comprises or is encoded by of a sequence as set forth as SEQ ID NO: 4: CTGCATGGATTCCATGTTCAT (miR-SOD-127). In some embodiments, the inhibitory nucleic acid comprises or is encoded by of a sequence as set forth as SEQ ID NO: 3: CTGCATCGATTCCATGTTCAT (miR-SOD-127). In some embodiments, the inhibitory nucleic acid is a mature miRNA that comprises SEQ ID NO: 3 and SEQ ID NO: 4. In some embodiments, SEQ ID NO: 3 is the guide strand of the mature miRNA and SEQ ID NO: 4 is the passenger strand (e.g., miRNA*) of the mature miRNA.

In some embodiments, the inhibitory nucleic acid is 5 to 30 bases in length (e.g., 10-30, 15-25, 19-22). The inhibitory nucleic acid may also be 10-50, or 5-50 bases length. For example, the inhibitory nucleic acid may be one of any of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 bases in length. In some embodiments, the inhibitory nucleic acid comprises or consists of a sequence of bases at least 80% or 90% complementary to, e.g., at least 5, 10, 15, 20, 25 or 30 bases of, or up to 30 or 40 bases of, the target nucleic acid, or comprises a sequence of bases with up to 6 mismatches over 10, 15, 20, 25 or 30 bases of the target nucleic acid.

In some embodiments, any one or more thymidine (T) nucleotides or uridine (U) nucleotides in a sequence provided herein may be replaced with any other nucleotide suitable for base pairing (e.g., via a Watson-Crick base pair) with an adenosine nucleotide. For example, T may be replaced with U. and U may be replaced with T. In some embodiments, inhibitory nucleic acids are provided that inhibit expression of genes in a cell of the central nervous system. In some embodiments, the cell is a neuron, astrocyte, or oligodendrocyte.

In some embodiments, an inhibitory nucleic acid is an miRNA. A “microRNA” or “miRNA” is a small non-coding RNA molecule capable of mediating transcriptional or post-translational gene silencing. Typically, miRNA is transcribed as a hairpin or stem-loop (e.g., having a self-complementarity, single-stranded backbone) duplex structure, referred to as a primary miRNA (pri-miRNA), which is enzymatically processed (e.g., by Drosha, DGCR8. Pasha, etc.) into a pre-miRNA. The length of a pri-miRNA can vary. In some embodiments, a pri-miRNA ranges from about 100 to about 5000 base pairs (e.g., about 100, about 200, about 500, about 1000, about 1200, about 1500, about 1800, or about 2000 base pairs) in length. In some embodiments, a pri-miRNA is greater than 200 base pairs in length (e.g., 2500, 5000, 7000, 9000, or more base pairs in length.

Pre-miRNA, which is also characterized by a hairpin or stem-loop duplex structure, can also vary in length. In some embodiments, pre-miRNA ranges in size from about 40 base pairs in length to about 500 base pairs in length. In some embodiments, pre-miRNA ranges in size from about 50 to 100 base pairs in length. In some embodiments, pre-miRNA ranges in size from about 50 to about 90 base pairs in length (e.g., about 50, about 52, about 54, about 56, about 58, about 60, about 62, about 64, about 66, about 68, about 70, about 72, about 74, about 76, about 78, about 80, about 82, about 84, about 86, about 88, or about 90 base pairs in length).

Generally, pre-miRNA is exported into the cytoplasm, and enzymatically processed by Dicer to first produce an imperfect miRNA/miRNA* duplex and then a single-stranded mature miRNA molecule, which is subsequently loaded into the RNA-induced silencing complex (RISC). Typically, a mature miRNA molecule ranges in size from about 19 to about 30 base pairs in length. In some embodiments, a mature miRNA molecule is about 19, about 20, about 21, about 22, about 23, about 24, about 25, about 26, about 27, about 28, about 29, or 30 base pairs in length. In some embodiments, an isolated nucleic acid of the disclosure comprises & sequence encoding a pri-miRNA, a pre-miRNA, or a mature miRNA comprising or encoded by a sequence set forth in SEQ ID NO: 4 (miR-SOD-127) and/or SEQ ID NO: 3.

In some aspects, the disclosure provides isolated nucleic acids and vectors (e.g., rAAV vectors) that encode one or more artificial miRNAs. As used herein “artificial miRNA” or “amiRNA” refers to an endogenous pri-miRNA or pre-miRNA (e.g., a miRNA backbone, which is a precursor miRNA capable of producing a functional mature miRNA), in which the miRNA “amiRNA” (e.g., passenger strand of the miRNA duplex) sequences have been replaced with corresponding amiRNA/amiRNA* sequences that direct highly efficient RNA silencing of the targeted gene, for example as described by Eamens et al. (2014). Methods Mol. Biol. 1062:211-224. For example, in some embodiments an artificial miRNA comprises a miR-155 pri-miRNA backbone into which a sequence encoding a mature SOD1-specific miRNA (e.g., SEQ ID NO: 3 and/or 4; miR-SOD-127) has been inserted in place of the endogenous miR-155 mature miRNA-encoding sequence. In some embodiments, miRNA (e.g., an artificial miRNA) as described by the disclosure comprises a miR-155 backbone sequence, a miR-30 backbone sequence, a mir-64 backbone sequence, a miR-106 backbone, a miR-21 backbone, a miR-1 backbone, a miR-451 backbone, a miR-126 backbone, or a miR-122 backbone sequence. In some embodiments, the inhibitory nucleic acid is a microRNA comprising a targeting sequence having flanking regions of miR 155 or miR-30.

It should be appreciated that an isolated nucleic acid or vector (e.g., rAAV vector), in some embodiments comprises a nucleic acid sequence encoding more than one (e.g., a plurality, such as 2, 3, 4, 5, 10, or more) miRNAs. In some embodiments, each of the more than one miRNAs targets (e.g., hybridizes or binds specifically to) the same target gene (e.g., an isolated nucleic acid encoding three unique miRNAs, where each miRNA targets the SOD1 gene). In some embodiments, each of the more than one miRNAs targets (e.g., hybridizes or binds specifically to) a different target gene.

### Isolated Nucleic Acids

In some aspects, the disclosure relates to isolated nucleic acidy comprising a first expression construct encoding a synthetic microRNA for inhibiting expression of endogenous SOD1 and a second expression construct to express exogenous SOD1 resistant to the synthetic microRNA (miRNA).

A “nucleic acid” sequence refers to a DNA or RNA sequence. In some embodiment, proteins and nucleic acids of the disclosure are isolated. As used herein, the term “isolated” means artificially produced. As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” refers to a protein or peptide that has been isolated from its natural environment or artificially produced (e.g., by chemical synthesis, by recombinant DNA technology, etc.).

Isolated nucleic acids of the disclosure typically comprise one or more regions that encode one or more inhibitory RNAs that target an endogenous mRNA (e.g., mRNA encoding endogenous wild-type SOD1 and/or endogenous mutant SOD1) of a subject. The isolated nucleic acids also typically comprise one or more regions that encode one or more exogenous mRNAs. The protein(s) encoded by the one or more exogenous mRNAs may or may not be different in sequence composition than the protein(s) encoded by the one or more endogenous mRNAs. For example, the one or more endogenous mRNAs may encode a wild-type and mutant version of a particular protein, such as may be the case when a subject is heterozygous for a particular mutation, and the exogenous mRNA may encode a wild-type mRNA of the same particular protein. In this case, typically the sequence of the exogenous mRNA and endogenous mRNA encoding the wild type protein are sufficiently different such that the exogenous mRNA is not targeted by the one or more inhibitory RNAs. This may be accomplished, for example, by introducing one or more silent mutations into the exogenous mRNA such that it encodes the same protein as the endogenous mRNA but has a different nucleic acid sequence. In this case, the exogenous mRNA may be referred to as “hardened.” Alternatively, the inhibitory RNA (e.g., miRNA) can target the 5′ and/or 3′ untranslated regions of the endogenous mRNA. These 5′ and/or 3′ regions can then be removed or replaced in the exogenous mRNA such that the exogenous miRNA is not targeted by the one or more inhibitory RNAs.

In another example, the one or more endogenous mRNAs may encode only mutant versions of a particular protein, such as may be the case when a subject is homozygous for a particular mutation, and the exogenous mRNA may encode a wild-type mRNA of the same particular protein. In this case, the sequence of the exogenous mRNA may be hardened as described above, or the one or more inhibitory RNAs may be designed to discriminate the mutated endogenous mRNA from the exogenous mRNA.

In some embodiments, the isolated nucleic acids typically comprise a first region that encodes one or more first inhibitory RNAs (e.g., miRNAs) comprising a nucleic acid having sufficient sequence complementary with an endogenous mRNA of a subject to hybridize with and inhibit expression of the endogenous miRNA (e.g., endogenous SOD1 mRNA). The isolated nucleic acids also typically include a second region encoding an exogenous mRNA (e.g., exogenous SOD1), in which the protein encoded by the exogenous mRNA has an amino acid sequence that is at least 95 ft identical to the first protein, in which the one or more first inhibitory RNAs do not comprise a nucleic acid having sufficient sequence complementary to hybridize with and inhibit expression of the exogenous mRNA. For example, the first region may be positioned at any suitable location. The first region may be positioned within an untranslated portion of the second region. The first region may be positioned in any untranslated portion of the nucleic acid, including, for example, an intron, a 5′ or 3′ untranslated region, etc.

A region comprising an inhibitory nucleic acid (e.g., a first region) may be positioned at any suitable location of the isolated nucleic acid. The region may be positioned in any untranslated portion of the nucleic acid, including, for example, an intron, a 5′ or 3′ untranslated region, etc.

In some cases, it may be desirable to position the region (e.g., the first region) upstream of the first codon of a nucleic acid sequence encoding a protein (such as a second region encoding an exogenous SOD1 protein coding sequence). For example, the region may be positioned between the first codon of a protein coding sequence and 2000 nucleotides upstream of the first codon. The region may be positioned between the first codon of a protein coding sequence and 1000 nucleotides upstream of the first codon. The region may be positioned between the first codon of a protein coding sequence and 500 nucleotides upstream of the first codon. The region may be positioned between the first codon of a protein coding sequence and 250 nucleotides upstream of the first codon. The region may be positioned between the first codon of a protein coding sequence and 150 nucleotides upstream of the first codon.

In some cases, it may be desirable to position the region (e.g., region encoding an inhibitory nucleic acid, such as a first region) upstream of the poly-A tall of a region encoding an exogenous SOD1 protein. For example, the region may be positioned between the first base of the poly-A tail and 2000 nucleotides upstream of the first base. The region may be positioned between the first base of the poly-A tail and 1000 nucleotides upstream of the first base. The region may be positioned between the first base of the poly-A tail and 500 nucleotides upstream of the first base. The region may be positioned between the first base of the poly-A tail and 250 nucleotides upstream of the first base. The region may be positioned between the first base of the poly-A tail and 150 nucleotides upstream of the first base. The region may be positioned between the first base of the poly-A tail and 100 nucleotides upstream of the first base. The region may be positioned between the first base of the poly-A tail and 50 nucleotides upstream of the first base. The region may be positioned between the first base of the poly-A tail and 20 nucleotides upstream of the first base. In some embodiments, the region is positioned between the last nucleotide base of a promoter sequence and the first nucleotide have of a poly-A tail sequence.

In some cases, a region encoding an inhibitory nucleic acid (e.g., a first region) may be positioned downstream of the last base of the poly-A tail of a region encoding an exogenous SOD1 protein. The region may be between the last base of the poly-A tail and a position 2000 nucleotides downstream of the last base. The region may be between the last base of the poly-A tail and a position 1000 nucleotides downstream of the last base. The region may be between the last base of the poly-A tail and a position 500 nucleotides downstream of the last base. The region may be between the last base of the poly-A tail and a position 250 nucleotides downstream of the fast base. The region may be between the last base of the poly-A tail and a position 150 nucleotides downstream of the last base.

It should be appreciated that in cases where an isolated nucleic acid encodes more than one miRNA, each miRNA may be positioned in any suitable location within the construct. For example, a nucleic acid encoding a first miRNA may be positioned in an intron of the region encoding an exogenous SOD1 protein and a nucleic acid sequence encoding a second miRNA may be positioned in another region (e.g., between the last codon of a protein coding sequence and the first base of the poly-A tail of the transgene).

In some embodiments, an isolated nucleic acid further comprises a nucleic acid sequence encoding one or more expression control sequences (e.g., a promoter, etc.). Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (I.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

A “promoter” refers to a DNA sequence recognized by the synthetic machinery of the cell, or introduced synthetic machinery, required to initiate the specific transcription of a gene. The phrases “operatively positioned.” “under control” or “under transcriptional control” means that the promoter is in the correct location and orientation in relation to the nucleic acid to control RNA polymerase initiation and expression of the gene.

For nucleic acids encoding proteins, a polyadenylation sequence generally is inserted following the transgene sequences and before the 3′ AAV ITR sequence. A rAAV construct useful in the present disclosure may also contain an intron, desirably located between the promoter/enhancer sequence and the transgene. One possible intron sequence is derived from SV-40, and is referred to as the SV-40 T intron sequence. Another vector element that may be used is an internal ribosome entry site (IRES). An IRES sequence is used to produce more than one polypeptide from a single gene transcript. An IRES sequence would be used to produce a protein that contain more than one polypeptide chains. Selection of these and other common vector elements are conventional and many such sequences are available [see. e.g., Sambrook et al., and reference, cited therein at, for example, pages 3.18 3.26 and 16.17 16.27 and Ausubel et al. Current Protocols in Molecular Biology, John Wiley & Sons, New York, 1989]. In some embodiments, a Foot and Mouth Disease Virus 2A sequence is included in polyprotein; this is a small peptide (approximately 18 amino acids in length) that has been shown to mediate the cleavage of polyproteins (Ryan, M D et al., EMBO, 1994; 933 Mattion, N M et al., 1 Virology, November 1996; p. 8124-8127; Furler, S et al., Gene Therapy, 2001; 8:864-873; and Halpin, C et al., The Plant Journal, 1999; 4:453-459). The cleavage activity of the 2A sequence has previously been demonstrated in artificial systems including plasmids and gene therapy vectors (AAV and retroviruses) (Ryan, M D et al., EMBO, 1994:4: 928-93; Mattion, N M et al., J Virology. November 1996; p. 8124-8127; Furler, S et al., Gene Therapy, 2001; 8:864-873; and Halpin, C et al., The Plant Journal, 1999:4: 453-459; de Felipe, P et al., Gene Therapy, 1999:6: 198-208; de Felipe, P et al., Human Gene Therapy, 2000; 11:1921-1931; and Klump, H et al., Gene Therapy, 2001; 8:811-817).

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al., Cell, 41:521-530 (1985)], the SV40 promoter, the dihydrofolate reductase promoter, the β-actin promoter (e.g., CBA promoter), the phosphoglycerol kinase (PGK) promoter, and the EF1α promoter [Invitrogen]. In some embodiments, a promoter is an enhanced chicken β-actin promoter (CAG promoter). In some embodiments, a promoter is a H1 promoter or a U6 promoter.

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only Inducible promoters and inducible systems are available from a variety of commercial sources, including, without limitation. Invitrogen, Clontech and Ariad. Many other systems have been described and can be readily selected by one of skill in the art. Examples of inducible promoters regulated by exogenously supplied promoters include the zine-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 547-5551 (1992), the tetracycline-inducible system (Gossen et al., Science, 268:1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997). Still other types of inducible promoters which may be useful in this context are those which are regulated by a specific physiological state, e.g. temperature, acute phase, a particular differentiation state of the cell, or in replicating cells only.

In another embodiment, the native promoter for SOD1 (e.g., SEQ ID NO: 16) will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (a-MHC) promoter, or a cardiac Troponin T (cTNT) promoter. Other exemplary promoters include Beta-actin promoter, hepatitis B virus core promoter. Sandig et al. Gene Ther., 3: 1002-9 (1996); alpha-fetoprotein (APP) promoter, Arbuthnot et al. Hum. Gene Ther., 7:1503-14 (1996), bone osteocalcin promoter (Stein et al. Mol. Biol. Rep., 24:185-96 (1997); bone sialoprotein promoter (Chen et al., J. Bone Miner. Res., 11:654-64 (1996)). CD2 promoter (Hansal et al., J. Immunol., 161:1063-8 (1998); immunoglobulin heavy chain promoter; T cell receptor α-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995), among others which will be apparent to the skilled artisan.

Aspects of the disclosure relate to an isolated nucleic acid comprising more than one promoter (e.g., 2, 3, 4, 5, or more promoters). For example, in the context of a construct having a transgene comprising a first region encoding an inhibitory RNA (e.g., miRNA) and a second region encoding an exogenous SOD1 protein, it may be desirable to drive expression of the inhibitory RNA encoding region using a first promoter sequence (e.g., a first promoter sequence operably linked to the inhibitory nucleic acid encoding region), and to drive expression of the exogenous SOD1-encoding region with a second promoter sequence (e.g., a second promoter sequence operably linked to the exogenous SOD1-encoding region). Generally, the first promoter sequence and the second promoter sequence can be the same promoter sequence or different promoter sequences. In some embodiments, the first promoter sequence (e.g., the promoter driving expression of the protein coding region) is a RNA polymerase III (poIIII) promoter sequence. Non-limiting examples of poIIII promoter sequences include U6 and H1 promoter sequences. In some embodiment, the second promoter sequence (e.g., the promoter sequence driving expression of the exogenous SOD1 RNA) is a RNA polymerase II (poIII) promoter sequence. Non-limiting examples of poIII promoter sequences include chicken beta actin promoter (CBA), T7, T3, SP6, RSV, and cytomegalovirus promoter sequences. In some embodiments, a poIIII promoter sequence drives expression of an inhibitory RNA (e.g., miRNA) encoding region. In some embodiments, a poIII promoter sequence drives expression of a protein coding region.

As described further below, the isolated nucleic acids may comprise inverted terminal repeats (ITR) of an AAV serotypes selected from the group consisting of: AAV1, AAV2, AAV5, AAV6, AAV6.2, AAV7, AAV8, AAV9, AAV10, AAV11 and variants thereof.

### Multicistronic Constructs

Some aspects of this invention provide multicistronic (e.g., bicistronic) expression constructs comprising two or more expression cassettes in various configurations.

In different embodiments, multicistronic (e.g., bicistronic) expression constructs are provided in which the expression cassettes are positioned in different ways. For example, in some embodiments, a multicistronic expression construct is provided in which a first expression cassette is positioned adjacent to a second expression cassette. In some embodiments, a multicistronic expression construct is provided in which a first expression cassette comprises an intron, and a second expression cassette is positioned within the intron of the first expression cassette. In some embodiments, the second expression cassette, positioned within an intron of the first expression cassette, comprises a promoter and a nucleic acid sequence encoding a gene product operatively linked to the promoter.

In different embodiments, multicistronic (e.g., bicistronic) expression constructs are provided in which the expression cassettes are oriented in different ways. For example, in some embodiments, a multicistronic expression construct is provided in which a first expression cassette is in the same orientation as a second expression cassette. In some embodiments, a multicistronic expression construct is provided comprising a first and a second expression cassette in opposite orientations.

The term “orientation” as used herein in connection with expression cassettes, refers to the directional characteristic of a given cassette or structure. In some embodiments, an expression cassette harbors a promoter 5′ of the encoding nucleic acid sequence, and transcription of the encoding nucleic acid sequence runs from the 5′ terminus to the 3″ terminus of the sense strand, making it a directional cassette (e.g. 5′-promoter/(intron)/encoding sequence-3′). Since virtually all expression cassettes are directional in this sense, those of skill in the art can easily determine the orientation of a given expression cassette in relation to a second nucleic acid structure, for example, a second expression cassette, a viral genome, or, if the cassette is comprised in an AAV construct, in relation to an AAV ITR.

For example, if a given nucleic acid construct comprises two expression cassettes in the configuration 5′-promoter 1/encoding sequence 1-promoter2/encoding sequence 2-3′,
<<<<<<<<<<<<<<<<<<<<<<< <<<<<<<<<<<<<<<<<<<<<<<
   the expression cassettes are in the same orientation, the arrows indicate the direction of transcription of each of the cassettes. For another example, if a given nucleic acid construct comprises a sense strand comprising two expression cassettes in the configuration
<<<<<<<<<<<<<<<<<<<<<<< <<<<<<<<<<<<<<<<<<<<<<<
   the expression cassettes are in opposite orientation to each other and, as indicated by the arrows, the direction of transcription of the expression cassettes, are opposed. In this example, the strand shown comprises the antisense strand of promoter 2 and encoding sequence 2.

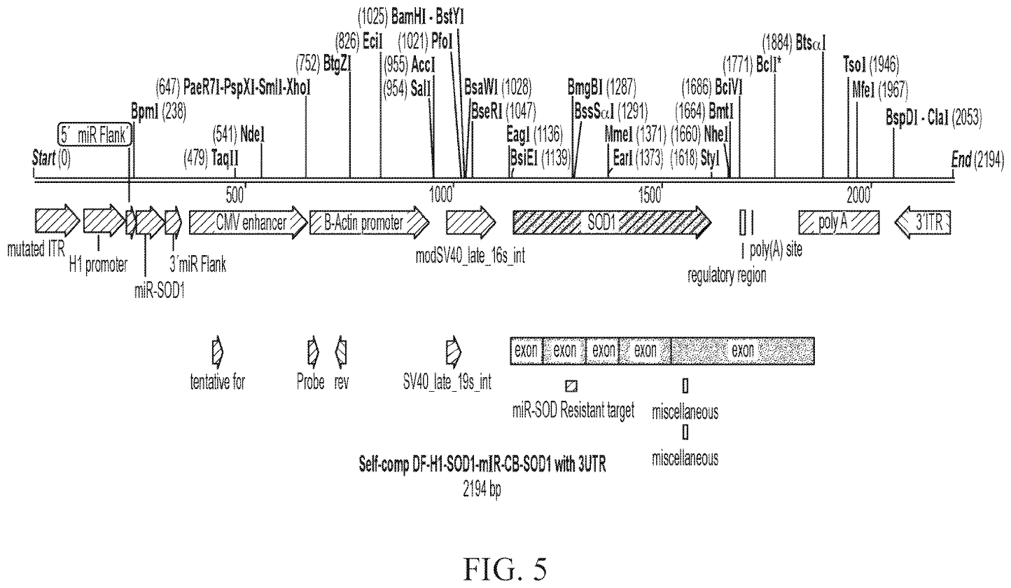

For another example, if an expression cassette is comprised in an AAV constrict, the cassette can either be in the same orientation as an AAV ITR (e.g., the structures depicted in FIG. 5, etc.), or in opposite orientation. AAV ITRs are directional. For example, the mutated 5′ITR exemplified in FIG. 5 would be in the same orientation as the H1 promoter/inhibitory RNA-encoding expression cassette, but in opposite orientation to the 3′ITR, if both ITRs and the expression cassette would be on the same nucleic acid strand.

rAAV Vectors

The isolated nucleic acids of the invention may be recombinant adeno-associated virus (AAV) vectors (rAAV vectors). In some embodiments, an isolated nucleic acid as described by the disclosure comprises a region (e.g., a first region) comprising a first adeno-associated virus (AAV) inverted terminal repeat (ITR), or a variant thereof. The isolated nucleic acid (e.g., the recombinant AAV vector) may be packaged into a capsid protein and administered to a subject and/or delivered to a selected target cell. “Recombinant AAV (rAAV) vectors” are typically composed of, at a minimum, a transgene and its regulatory sequences, and 5′ and 3′ AAV inverted terminal repeats (ITRs). The transgene may comprise, as disclosed elsewhere herein, one or more regions that encode one or more inhibitory RNAs (e.g., miRNAs) comprising a nucleic acid that targets an endogenous mRNA of a subject. The transgene may also comprise a region encoding, for example, a protein and/or an expression control sequence (e.g., a poly-A tail), as described elsewhere in the disclosure.

Generally, ITR sequences are about 145 base pairs (bp) in length. Preferably, substantially the entire sequences encoding the ITRs are used in the molecule, although some degree of miner modification of these sequences is permissible. The ability to modify these ITR sequences is within the skill of the art. (See, e.g., texts such as Sambrook et al., “Molecular Cloning, A Laboratory Manual”, 2d ed., Cold Spring Harbor Laboratory, New York (1989); and K. Fisher et al., J Virol., 70:520 532 (1996). An example of such a molecule employed in the present invention is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types. In some embodiments, the Isolated nucleic acid (e.g., the rAAV vector) comprises at least one ITR having a serotype selected from AAV1, AAV2, AAV5, AAV6, AAV6.2, AAV7, AAV8, AAV9, AAV10, AAV11, and variants thereof. In some embodiments, the isolated nucleic acid comprises a region (e.g., a first region) encoding an AAV2 ITR.

In some embodiments, the isolated nucleic acid further comprises a region (e.g., a second region, a third region, a fourth region, etc.) comprising a second AAV ITR. In some embodiment, the second AAV ITR has a serotype selected from AAV1, AAV2. AAV5, AAV6, AAV6.2, AAV7, AAV8, AAV9, AAV10, AAV11, and variants thereof. In some embodiments, the second ITR is a mutant ITR that lacks a functional terminal resolution site (TRS). The term “lacking a terminal resolution site” can refer to an AAV ITR that comprises a mutation (e.g., a sense mutation such as a non-synonymous mutation, or missense mutation) that abrogates the function of the terminal resolution site (TRS) of the ITR, or to a truncated AAV TR that lacks #nucleic acid sequence encoding a functional TRS (e.g., a ΔTRS ITR). Without wishing to be bound by any particular theory, a rAAV vector comprising an ITR lacking a functional TRS produces a self-complementary rAAV vector, for example as described by McCarthy (2008) Molecular Therapy 16 (10): 1648-1656.

In addition to the major elements identified above for the recombinant AAV vector, the vector also includes conventional control elements which are operably linked with elements of the transgene in a manner that permits its transcription, translation and/or expression in a cell transfected with the vector or infected with the virus produced by the invention. As used herein. “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A number of expression control sequences, including promoters which are native, constitutive, inducible and/or tissue-specific, are known in the art and may be utilized.

As used herein, a nucleic acid sequence (e.g., coding sequence) and regulatory sequences are said to be operably linked when they are covalently linked in such a way as to place the expression or transcription of the nucleic acid sequence under the influence of control of the regulatory sequences. If it is desired that the nucleic acid sequences be translated into a functional protein, two DNA sequences are said to be operably linked if induction of a promoter in the S′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly two or more coding regions are operably linked when they are linked in such a way that their transcription from a common promoter results in the expression of two or more proteins having been translated in frame. In some embodiments, operably linked coding sequences yield a fusion protein.

Recombinant Adeno-Associated Viruses (rAAVs)

In some aspects, the disclosure provides isolated AAVs. As used herein with respect to AAVs, the term “isolated” refers to an AAV that has been artificially produced or obtained. Isolated AA Vs may be produced using recombinant methods. Such AA Vs are referred to herein as “recombinant AAVs”. Recombinant AAVs (rAAVs) preferably have tissue-specific targeting capabilities, such that a nuclease and/or transgene of the rAAV will be delivered specifically to one or more predetermined tissue(s). The AAV capsid is an important element in determining these tissue-specific targeting capabilities. Thus, an rAAV having a capsid appropriate for the tissue being targeted can be selected.

Methods for obtaining recombinant AAVs having a desired capsid protein are well known in the art. (See, for example, US 2003/0138772), the contents of which are incorporated herein by reference in their entirety). Typically the methods involve culturing a host cell which contains a nucleic acid sequence encoding an AAV capsid protein; a functional rep gene; recombinant AAV vector composed of, AAV inverted terminal repeats (ITRs) and a transgene; and sufficient helper functions to permit packaging of the recombinant AAV vector into the AAV capsid proteins. In some embodiments, capsid proteins are structural proteins encoded by the cap gene of an AAV. AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 Da, about 72 kDa and about 62 kDa. In some embodiments, upon translation, capsid proteins form a spherical 60-mer protein shell around the viral genome. In some embodiments, the functions of the capsid proteins are to protect the viral genome, deliver the genome and interact with the host. In some aspects, capsid proteins deliver the viral genome to a host in a tissue specific manner.

In some embodiments, an AAV capsid protein is of an AAV serotype selected from the group consisting of AAV2. AAV3. AAV4, AAV5. AAV6, AAV8, AAVrh8, AAV9, AAV10. AAVrh.10, AAV AAV.PHB, and variants of any of the foregoing. In some embodiments, an AAV capsid protein is of a serotype derived from a non-human primate, for example AAVrh10 serotype. In some embodiments, an AAV capsid protein is of an AAV9 serotype.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one of more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

In some embodiments, the instant disclosure relates to a host cell containing a nucleic acid that comprises sequence encoding an inhibitory nucleic acid targeting endogenous SOD1 and a sequence encoding an exogenous protein (e.g., exogenous SOD1 protein, optionally “hardened” exogenous SOD1 protein). In some embodiments, the instant disclosure relates to a composition comprising the host cell described above. In some embodiments, the composition comprising the host cell above further comprises a cryopreservative.

The recombinant AAV vector, rep sequences, cap sequences, and helper functions required for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press. Cold Spring Harbor, N.Y. Similarly, methods of generating rAAV virions are well known and the selection of a suitable method is not a limitation on the present disclosure. See. e.g., K. Fisher et al., J. Virol., 70:520-532 (1993) and U.S. Pat. No. 5,478,745.

In some embodiments, recombinant AAV's may be produced using the triple transfection method (described in detail in U.S. Pat. No. 6,001,650). Typically, the recombinant AAV's are produced by transfecting a host cell with an recombinant AAV vector (comprising a transgene) to be packaged into AAV particles, an AAV helper function vector, and an accessory function vector. An AAV helper function vector encodes the “AAV helper function” sequences (i.e., rep and cap), which function in trans for productive AAV replication and encapsidation. Preferably, the AAV helper function vector supports efficient AAV vector production without generating any detectable wild-type AAV virions (i.e., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. See. e.g., Graham et al. (1973) Virology, 52:456, Sambrook et al. (1989) Molecular Cloning, a laboratory manual. Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment to be transcribed is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned.” “under control” or “under transcriptional control” means that the promoter is in the correct location and orientation in relation to the nucleic acid to control RNA polymerase initiation and expression of the gene. The term “expression vector or construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product or functional RNA (e.g., guide RNA) from a transcribed gene.

The foregoing methods for packaging recombinant vectors in desired AAV capsids to produce the (AAVs of the disclosure are not meant to be limiting and other suitable methods will be apparent to the skilled artisan.

### Modes of Administration

Isolated nucleic acids and rAAVs of the disclosure may be delivered to a cell or subject in compositions according to any appropriate methods known in the art. For example, an rAAV, preferably suspended in a physiologically compatible carrier (i.e., in a composition), may be administered to a subject, i.e. host animal, such as a human, mouse, rat, cat, dog, sheep, rabbit, horse, cow, goat, pig, guinea pig, hamster, chicken, turkey, non-human primate (e.g., Macaque). In some embodiments a host animal does not include a human.

Delivery of the rAAVs to a mammalian subject may be by, for example, intramuscular injection or by administration into the bloodstream of the mammalian subject. Administration into the bloodstream may be by injection into a vein, an artery, or any other vascular conduit. In some embodiments, the rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer the virions into the vasculature of an isolated limb to potentially enhance transduction into muscle cells or tissue. Moreover, in certain instances, it may be desirable to deliver the virions to the CNS of a subject. By “CNS” is meant all cells and tissue of the brain and spinal cord of a vertebrate. Thus, the term includes, but is not limited to, neuronal cells, glial cells, astrocytes, cerebrospinal fluid (CSF), interstitial spaces, bone, cartilage and the like. Recombinant AAV's may be delivered directly to the CNS or brain by injection into, e.g., the ventricular region, as well as to the striatom (e.g., the candate nucleus or putamen of the striatum), spinal cord and neuromuscular junction, or cerebellar lobule, with a needle, catheter or related device, using neurosurgical techniques known in the art, such as by stereotactic injection (see, e.g., Stein et al., 3 Virol 73:3424-3429, 1999; Davidson et al., PNAS 97:3428-3432, 2000; Davidson et al., Nat. Genet. 3:219-223, 1993; and Alisky and Davidson. Hum. Gene Ther. 11:2315-2329, 2000). In some embodiment, rAAV as described in the disclosure are administered by intravenous injection. In some embodiments, the rAAV are administered by intracerebral injection. In some embodiments, the rAAV are administered by intrathecal injection. In some embodiments, the rAAV are administered by intrastriatal injection. In some embodiments, the rAAV are delivered by intracranial injection. In some embodiments, the rAAV are delivered by cisterna magna injection. In some embodiments, the rAAV are delivered by cerebral lateral ventricle injection. Aspects of the instant disclosure relate to compositions comprising a recombinant AAV comprising a capsid protein and a nucleic acid encoding a transgene, wherein the transgene comprises a nucleic acid sequence encoding one or more miRNAs. In some embodiments, each miRNA comprises or is encoded by a sequence set forth in SEQ ID NO: 3 and/or 4 (miR-SOD-127). In some embodiments, each miRNA comprises or is encoded by a sequence set forth in SEQ ID NO: 5 and/or 6. In some embodiments, the nucleic acid further comprises AAV ITRs.

In some embodiments, the rAAV comprises an rAAV vector represented by the sequence set forth in any one of SEQ ID NO: 8-15 (AAV vector sequences), or a portion thereof. In some embodiments, a composition further comprises a pharmaceutically acceptable carrier.

The compositions of the disclosure may comprise an rAAV alone, or in combination with one or more other viruses (e.g., a second rAAV encoding having one or more different transgenes). In some embodiments, a composition comprises 1, 2, 3, 4, 5, 6, 7, 8, 9, 10, or more different rAAVs each having one or more different transgenes.

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Optionally, the composition of the disclosure may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The rAAVs are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., intraportal delivery to the liver), oral, inhalation (including intranasal and intratracheal delivery), intraocular, intravenous, intramuscular, subcutaneous, intradermal, intratumoral, and other parental routes of administration. Routes of administration may be combined, if desired.

The dose of rAAV virions required to achieve a particular “therapeutic effect,” e.g., the units of dose in genome copies/per kilogram of body weight (GC/kg), will vary based on several factors including, but not limited to: the route of rAAV virion administration, the level of gene or RNA expression required to achieve a therapeutic effect, the specific disease or disorder being treated, and the stability of the gene or RNA product. One of skill in the art can readily determine a rAAV virion dose range to treat a patient having a particular disease or disorder based on the aforementioned factors, as well as other factors that are well known in the art.

An effective amount of an rAAV is an amount sufficient to target infect an animal, target a desired tissue. In some embodiments, an effective amount of an rAAV is an amount sufficient to produce a stable somatic transgenic animal model. The effective amount will depend primarily on factors such as the species, age, weight, health of the subject, and the tissue to be targeted, and may thus vary among animal and tissue. For example, an effective amount of the rAAV is generally in the range of from about 1 ml to about 100 ml of solution containing from about 10⁹ to 10¹⁶ genome copies. In some cases, a dosage between about 10¹¹ to 10¹³ rAAV genome copies is appropriate. In certain embodiments, 10¹² or 10¹³ rAAV genome copies is effective to target CNS tissue. In some cases, stable transgenic animals are produced by multiple doses of an rAAV.

In some embodiments, a dose of rAAV is administered to a subject no more than once per calendar day (e.g., a 24-hour period). In some embodiments, a dose of rAAV is administered to a subject no more than once per 2, 3, 4, 5, 6, or 7 calendar days. In some embodiments, a dose of rAAV is administered to a subject no more than once per calendar week (e.g., 7 calendar days). In some embodiments, a dose of rAAV is administered to a subject no more than bi-weekly (e.g., once in a two calendar week period). In some embodiments, a dose of rAAV is administered to a subject no more than once per calendar month (e.g., once in 30 calendar days). In some embodiments, a dese of rAAV is administered to a subject no more than once per six calendar months. In some embodiments, a dose of rAAV is administered to a subject no more than once per calendar year (e.g., 365 days or 366 days in a leap year).

In some embodiments, rAAV compositions are formulated to reduce aggregation of AAV particles in the composition, particularly where high rAAV concentrations are present (e.g. ˜10¹³ (GC/ml or more). Methods for reducing aggregation of rAAVs are well known in the art and, include, for example, addition of surfactants, pH adjustment, salt concentration adjustment, etc. (See, e.g., Wright F R, et al., Molecular Therapy (2005) 12, 171-178, the contents of which are incorporated herein by reference.)

Formulation of pharmaceutically-acceptable excipients and carrier solutions is well-known to those of skill in the art, as is the development of suitable dosing and treatment regimens for using the particular compositions described herein in a variety of treatment regimens.

Typically, these formulations may contain at least about 0.19% of the active compound or more, although the percentage of the active ingredient(s) may, of course, be varied and may conveniently be between about 1 or 29% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the rAAV-based therapeutic constructs in suitably formulated pharmaceutical compositions disclosed herein either subcutaneously, intraopancreatically, intranasally, parenterally, intravenously, intramuscularly, intrathecally, or orally, intraperitoneally, or by inhalation. In some embodiments, the administration modalities as described in U.S. Pat. Nos. 5,543,158; 5,641,515 and 5,399,363 (each specifically incorporated herein by reference in its entirety) may be used to deliver rAAVs. In some embodiments, a preferred mode of administration is by portal vein injection.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile filtered solution thereof.

The rAAV compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the rAAV vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 am to 4 μm. Sonication of MLVs results in the formation of small unilamellar vesicles (SUVs) with diameters in the range of 200 to 500 Å, containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

### Methods of Use

Methods are provided herein for inhibiting the expression of genes that are associated with FTD and/or ALS, such as SOD1. In some embodiments, methods described by the disclosure are useful for treating a subject having or suspected of having ALS and/or FTD. As used herein “treat” or “treating” refers to (a) preventing or delaying onset of neurodegenerative disease (e.g., ALS/FTD, etc.); (b) reducing severity of ALS/FTD; (c) reducing or preventing development of symptoms characteristic of ALS/FTD; (d) and/or preventing worsening of symptoms characteristic of ALS/FTD.

In some embodiments, methods are provided for inhibiting endogenous SOD1 protein expression in a subject (e.g., the central nervous system (CNS) of a subject). In some embodiments, the methods involve administering to the subject (e.g., administering to the CNS of the subject) an isolated nucleic acid or rAAV engineered to express an inhibitory nucleic acid that targets endogenous SOD1 mRNA and an exogenous SOD1 mRNA transcript that is resistant to the inhibitory nucleic acid. In some embodiments, the subject has or is suspected of having FTD or ALS (e.g., has been identified, for example by diagnostic DNA testing, as having a SOD1 gene having one or more mutations leading to a toxic gain of function and/or exhibits one or more signs or symptoms of ALS). In some embodiments, the methods involve administering to the subject an effective amount of a recombinant adeno associated virus ((AAV) harboring a nucleic acid that is engineered to express, in a cell of the subject, an inhibitory nucleic acid that targets endogenous SOD1 mRNA. In some embodiments, the inhibitory nucleic acid comprises or is encoded by a sequence as set forth in SEQ ID NO: 3 (GACGTACCTAAGGTACAAGTA) and/or 4 (miR-SOD-127). In some embodiments, the inhibitory nucleic acid comprises or is encoded by a sequence as set forth in SEQ ID NO: 5 and/or 6.

In some embodiments, methods are provided for inhibiting SOD1 expression in a cell. In some embodiments, the methods involve delivering to the cell an isolated nucleic acid or rAAV as described by the disclosure, wherein the inhibitory RNA is an miRNA that comprises or is encoded by 5, 6, 7, 8, 9, 10, 11, 12, 13, 14, 15, 16, 17, 18, 19, 20 or 21 consecutive nucleotides of a sequence set forth in SEQ ID NO: 3 (GACGTACCTAAGCTACAAGTA) and/or 4 (CTCCATCGATTCCATGTTCAT), or of a complementary sequence of that sequence.

In accordance with the foregoing, certain methods provided herein involve administering to a subject an effective amount of a recombinant Adeno-Associated Virus (rAAV) harboring any of the recombinant nucleic acids disclosed herein. In general, the “effective amount” of a rAAV refers to an amount sufficient to elicit the desired biological response. In some embodiments, the effective amount refers to the amount of rAAV effective for transducing a cell or tissue ex vivo. In other embodiments, the effective amount refers to the amount effective for direct administration of rAAV to a subject. As will be appreciated by those of ordinary skill in this art, the effective amount of the recombinant AAV of the invention varies depending on such factors as the desired biological endpoint, the pharmacokinetics of the expression products, the condition being treated, the mode of administration, and the subject. Typically, the rAAV is administered with a pharmaceutically acceptable carrier, as described elsewhere in this disclosure.

In some instances, after administration of the rAAV at least one clinical outcome parameter or biomarker (e.g., intranuclear (AC; RNA foci, RAN-protein expression, etc.) associated with the FTD or ALS is evaluated in the subject. Typically, the clinical outcome parameter or biomarker evaluated after administration of the rAAV is compared with the clinical outcome parameter or biomarker determined at a time prior to administration of the rAAV to determine effectiveness of the rAAV. Often an improvement in the clinical outcome parameter or biomarker after administration of the rAAV indicates effectiveness of the rAAV. Any appropriate clinical outcome parameter or biomarker may be used. Typically, the clinical outcome parameter or biomarker is indicative of the one or more symptoms of an FTD or ALS. For example, in some embodiments, the clinical outcome parameter or biomarker may be endogenous SOD1 expression, memory loss, or presence or absence of movement disorders such as unsteadiness, rigidity, slowness, twitches, muscle weakness or difficulty swallowing, speech and language difficulties, twitching (fasciculation) and cramping of muscles, including those in the hands and feet.

### Kits and Related Compositions

The recombinant nucleic acids, compositions, rAAV vectors, rAAVs, etc. described herein may, in some embodiments, be assembled into pharmaceutical or diagnostic or research kits to facilitate their use in therapeutic, diagnostic or research applications. A kit may include one or more containers housing the components of the invention and instructions for use. Specifically, such kits may include one or more agents described herein, along with instructions describing the intended application and the proper use of these agents. In certain embodiments agents in a kit may be in a pharmaceutical formulation and dosage suitable for a particular application and for a method of administration of the agents. Kits for research purposes may contain the components in appropriate concentrations or quantities for running various experiments.

The kit may be designed to facilitate use of the methods described herein by researchers and can take many forms. Each of the compositions of the kit, where applicable, may be provided in liquid form (e.g., in solution), or in solid form. (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the invention. Instructions also can include any oral or electronic instructions provided in any manner such that a user will clearly recognize that the instructions are to be associated with the kit, for example, audiovisual (e.g., videotape, DVD, etc.). Internet, and/or web-based communications, etc. The written instructions may be in a form prescribed by a governmental agency regulating the manufacture, use or sale of pharmaceuticals or biological products, which instructions can also reflects approval by the agency of manufacture, use or sale for animal administration.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be in the form of a liquid, gel or solid (powder). The agents may be prepared sterilely, packaged in syringe and shipped refrigerated. Alternatively it may be housed in a vial or other container for storage. A second container may have other agents prepared sterilely. Alternatively the kit may include the active agents premixed and shipped in a syringe, vial, tube, or other container. The kit may have one or more or all of the components required to administer the agents to a subject, such as a syringe, topical application devices, or IV needle tubing and bag.

Exemplary embodiments of the invention will be described in more detail by the following examples. These embodiments are exemplary of the invention, which one skilled in the art will recognize is not limited to the exemplary embodiments.

### EXAMPLES

### Example 1

This example describes dual expression gene therapy vectors that couple delivery of (1) a first construct engineered to express synthetic microRNA to silence expression of endogenous cytosolic Cu/Zn superoxide dismutase (SOD1) activity with (2) a second construct engineered to express wildtype SOD1 resistant to the synthetic microRNA.

The rationale for coupling SOD1 silencing via AAVrh10-antiSOD1-miRNA with expression of WT SOD1 resistant to the synthetic microRNA is based on two factors. First, the dismutation activity of the SOD1 protein has neuroprotective properties. Second, the tissues (and specifically the motor neurons) of ALS cases in which SOD1 is silenced are not normal, precisely because they express both wild-type (WT) and mutant SOD1. Indeed, when SOD1 silencing studies are initiated after disease onset, the motor neurons (and some non-neuronal cells) are already observed to be manifestly pathological. In this situation, to eliminate the SOD1 dismutation activity conferred by the WT SOD1 molecule (and also dismutation activity that can arise from some mutant SOD1 proteins) is also to eliminate potentially neuroprotective influences conferred by that activity. The net effect on the cells therefore reflects a balance of two opposite factors: (a) silencing the mutant protein and its neurotoxicity versus (b) eliminating the neuroprotective influence of the SOD1 dismutation activity. In a sick motor neuron, it is conceivable that the net effect may be to further compromise the viability of the targeted cell, despite simultaneous reduction in levels of the mutant protein. Consistent with this observation, it is noted that while mice devoid of intrinsic SOD1 activity do not develop fulminant ALS during normal development, their motor neurons are highly susceptible to superimposed injury; facial nerves injury in those SOD1-negative mice leads to much more extensive loss of facial nerves than in WT mice. Moreover, late in life these SOD1-negative mice have been observed to develop a slowly progressive, late-onset motor neuronopathy. The dual expression gene constructs described by the disclosure address the challenge of loss of neuroprotective activity from SOD1 dismutation. The arrangement of gene expression cassettes in constructs of the disclosure allows for normal levels of SOD1 dismutation activity (e.g., expression of WT SOD1) even with total silencing of both WT and mutant endogenous SOD1 alleles. Thus, the net effect of the constructs described herein is a reduction in levels of the mutant SOD1 protein (but not WT SOD1 protein), which is beneficial in SOD1-mediated ALS.

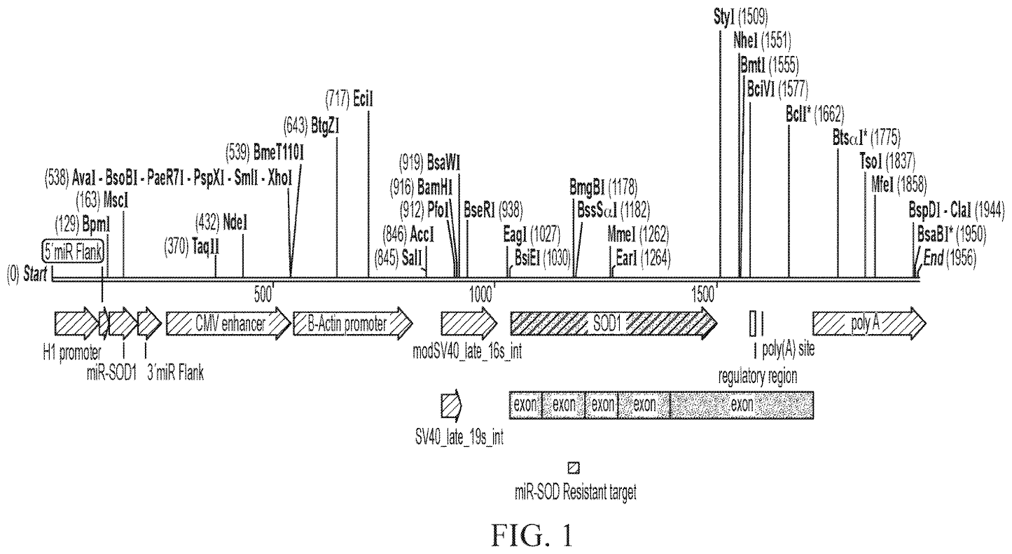

Dual expression constructs of the disclosure are constructed as follows: an AAV construct that expresses both an artificial miRNAs that targets SOD1 and a SOD1 eDNA that has silent base pair modification that makes it resistant to the artificial miRNA is produced. This construct simultaneously allows silencing of mutant SOD1 and augmented expression of wildtype SOD1 from a single AAV vector. In some embodiments, the construct is bicistronic as shown in FIG. 1, where the construct has 2 promoters; for example, anti-SOD1 expression is driven by a H1 promoter and SOD1 cDNA expression is driven by a CBA promoter. The anti-SOD1-miR expression can also be driven by another Pol III promoter, such as U6 promoter, or a Pol II promoter to restrict expression of the miRNA to a specific cell or organ type. The second portion of the constructs typically has a Pol II promoter (e.g., CBA in FIG. 1) expressing the miRNA resistant SOD1 cDNA. This second promoter can also be the endogenous SOD1 promotor, or another promoter such as the synapsin promoter if restricted expression of the SOD1 cDNA to specific cell population is desired.

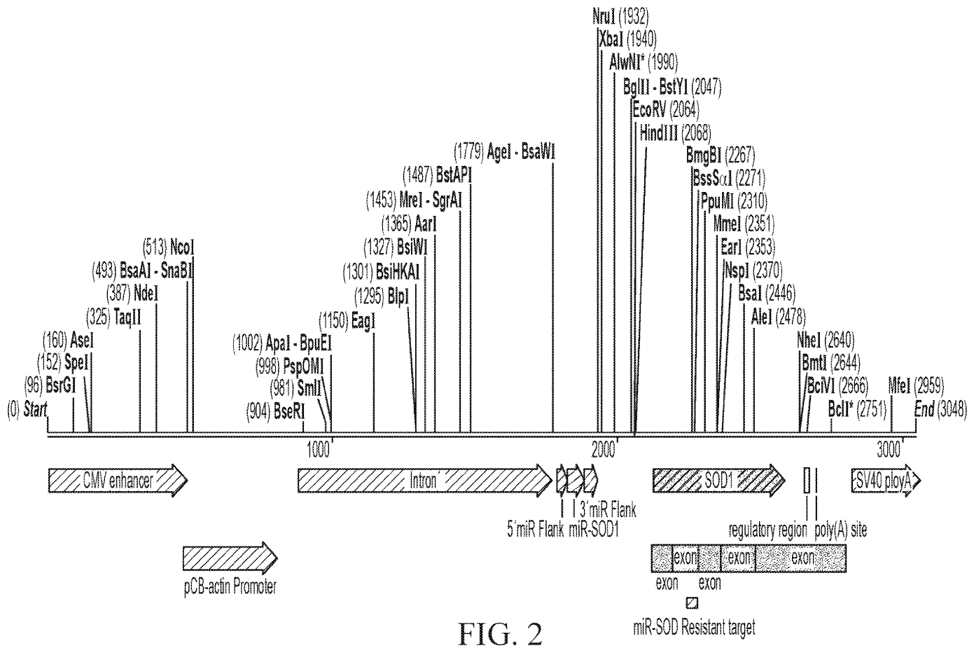

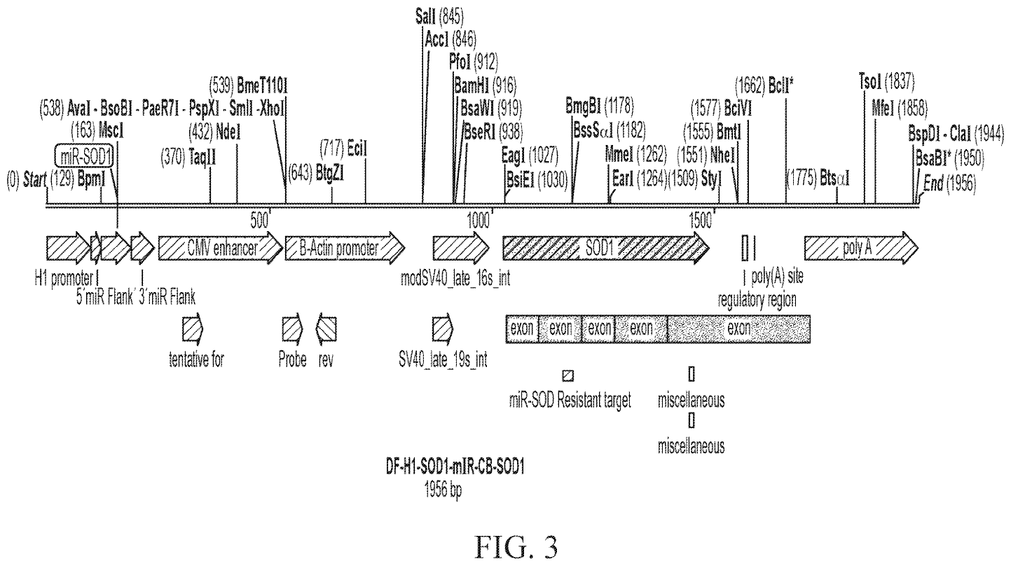

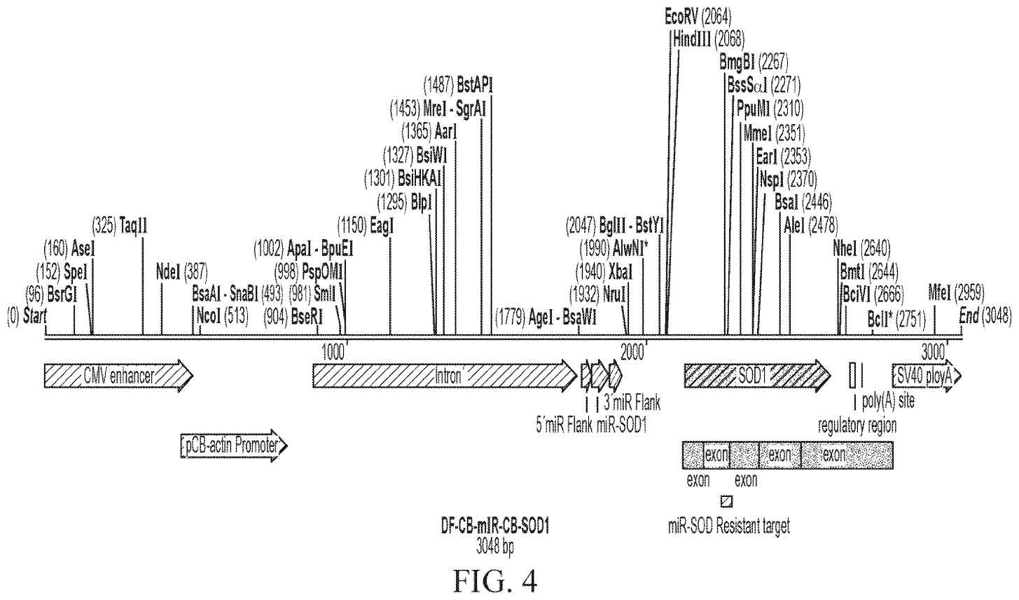

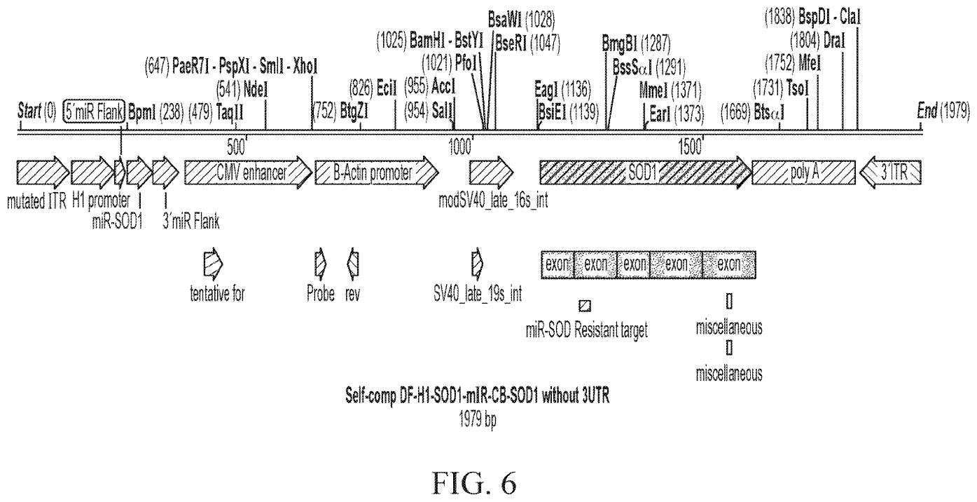

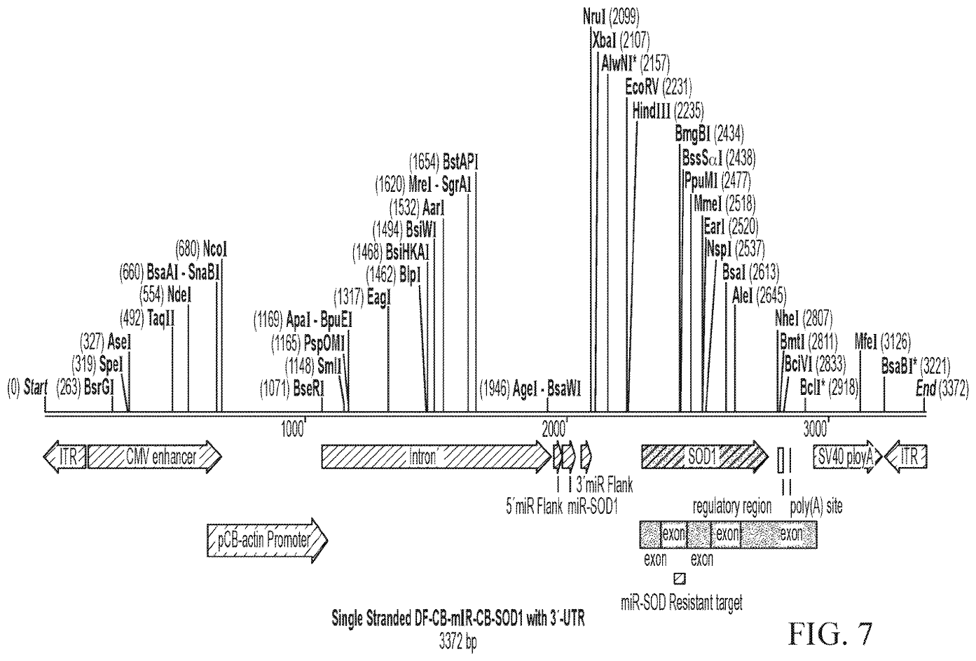

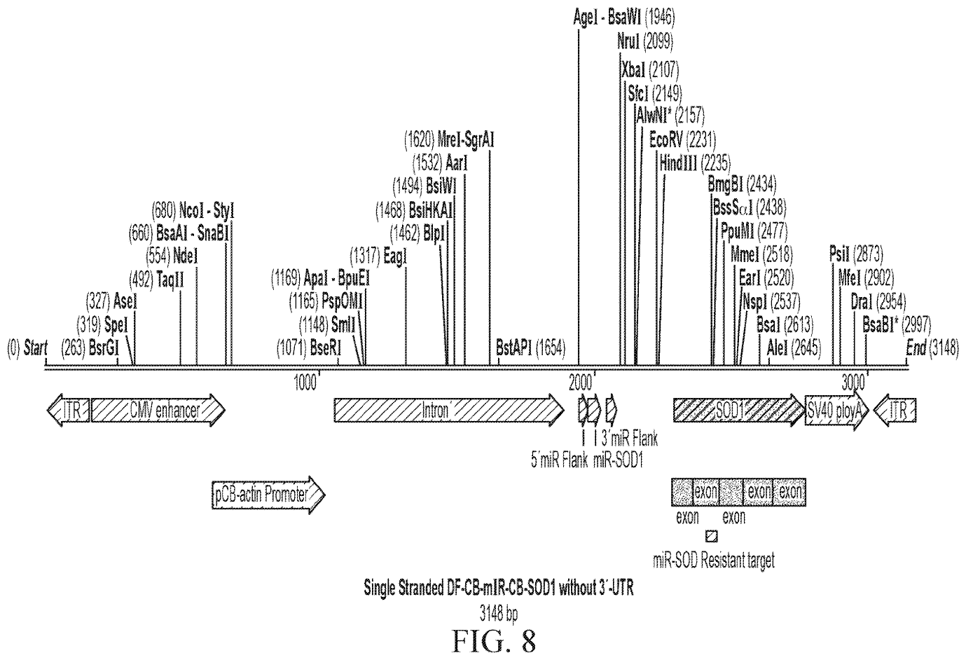

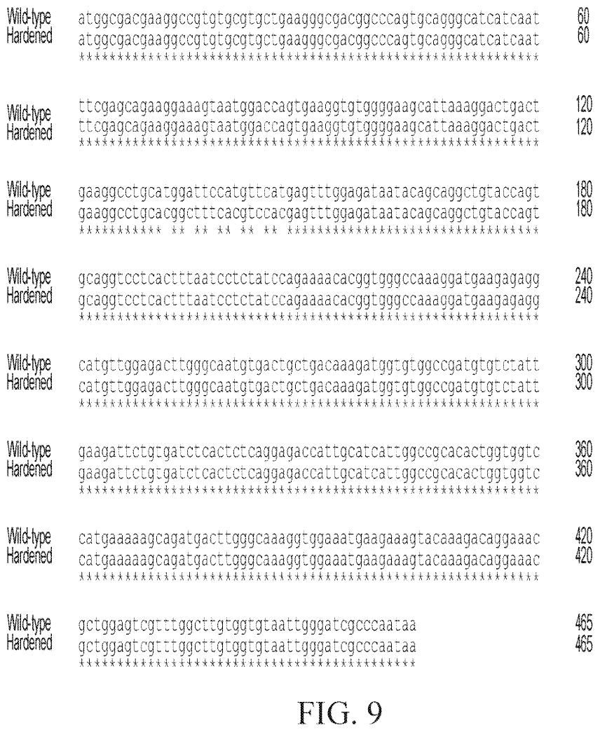

In some embodiments, the dual function vector is a single pol II promoter (e.g., CBA) expressing both the artificial miR and the miR-resistant cDNA, as shown in FIG. 2. In this embodiment, the anti-SOD1-miR can be expressed from an intron within the SOD1 eDNA expression cassette, or alternatively as part of the 3′UTR (or 5′ UTR) of the mIR-resistant SOD1 cDNA expression cassette. Additional non-limiting examples of dual function vector constructs are shown in FIGS. 3-8 and described in SEQ ID NOs: 8-15. FIG. 9 shows a nucleic acid sequence alignment of wild-type SOD1 coding sequence (SEQ ID NO: 1) with an example of a “hardened” SOD1 coding sequence (SEQ ID NO: 7).

SEQUENCES

> Human SOD1 coding sequence (NCBI Ref. NM_000454.4)(SEQ ID NO: 1)
ATGGCGACGAAGGCCGTGTGCGTGCTGAAGGGCGACGGCCCAGTGCAGGGCATCAT
CAATTTCGAGCAGAAGGAAAGTAATGGACCAGTGAAGGTGTGGGGAAGCATTAAA
GGACTGACTGAAGGCCTGCATGGATTCCATGTTCATGAGTTTGGAGATAATACAGC
AGGCTGTACCAGTGCAGGTCCTCACTTTAATCCTCTATCCAGAAAACACGGTGGGCC
AAAGGATGAAGAGAGGCATGTTGGAGACTTGGGCAATGTGACTGCTGACAAAGAT
GGTGTGGCCGATGTGTCTATTGAAGATTCTGTGATCTCACTCTCAGGAGACCATTGC
ATCATTGGCCGCACACTGGTGGTCCATGAAAAAGCAGATGACTTGGGCAAAGGTGG
AAATGAAGAAAGTACAAAGACAGGAAACGCTGGAAGTCGTTTGGCTTGTGGTGTAA
TTGGGATGGCCCAATAA
>SOD1 miR target sequence 5′-3′; note in some embodiments, “T” is replaced with “U” (SEQ
ID NO: 2)
CTGCATGGATTCCATGTTCAT
>SOD1 miR mature miRNA 3′-5′; note in some embodiments, “T” is replaced with “U” (SEQ
ID NO: 3)
GACGTACCTAAGGTACAAGTA
>SOD-miR-127 mature miRNA 5′-3′; note in some embodiments, “T” is replaced with “U”
(SEQ ID NO: 4)
CTGCATGGATTCCATGTTCAT
>miR-SOD1 5′-3′ strand (SEQ ID NO: 5); note in some embodiments, “T” is replaced with “U”
TGCTGATGAACATGGAATCCATGCAGGTTTTGGCCACTGACTGACCTGCATGGTCCA
TGTTCAT
>miR-SOD1 3′-5′ strand (SEQ ID NO: 6); note in some embodiments, “T” is replaced with “U”
ATGAACATGGACCATGCAGGTCAGTCAGTGGCCAAAACCTGCATGGATTCCATGTT
CATCAGCA
> Hardened SOD1 coding sequence (SEQ ID NO: 7); silent base pair mutations relative to wild-
type SOD1 coding sequence in bold
ATGGCGACGAAGGCCGTGTGCGTGCTGAAGGGCGACGGCCCAGTGCAGGGCATCAT
CAATTTCGAGCAGAAGGAAAGTAATGGACCAGTGAAGGTGTGGGGAAGCATTAAA
GGACTGACTGAAGGCCTGCACGGCTTTCACGTCCACGAGTTTGGAGATAATACAGC
AGGCTGTACCAGTGCAGGTCCTCACTTTAATCCTCTATCCAGAAAACACGGTGGGCC
AAAGGATGAAGAGAGGCATGTTGGAGACTTGGGCAATGTGACTGCTGACAAAGAT
GGTGTGGCCGATGTGTCTATTGAAGATTCTGTGATCTCACTCTCAGGAGACCATTGC
ATCATTGGCCGCACACTGGTGGTCCATGAAAAAGCAGATGACTTGGGCAAAGGTGG
AAATGAAGAAAGTACAAAGACAGGAAACGCTGGAAGTCGTTTGGCTTGTGGTGTAA
TTGGGATCGCCCAATAA
> Sequence for Bicistronic H1 -miR and CB-Sod1 (SEQ ID NO: 8)
CTCTGGTCGTTACATAACTTACGGTAAATGGCCCGCCTGGCTGACCGGCCAACGACC
CCCGCCCATTGACGTCAATAATGACGTATGTTCCCATAGTAACGCCAATAGGGACTT
TCCATTGACGTCAATGGGTGGAGTATTTACGGTAAACTGCCCACTTGGCAGTACATC
AAGTGTATCATATGCCAAGTACGCCCCCTATTGACGTCAATGACGGTAAATGGCCC
GCCTGGCATTATGCCCAGTACATGACCTTATGGGACTTTCCTACTTGGCAGTACATC
TACTCGAGGCCACGTTCTGCTTCACTCTCCCCATCTCCCCCCCCTCCCCACCCCCAAT
TTTGTATTTATTTATTTTTTAATTATTTTGTGCAGCGATGGGGGCGGGGGGGGGGGG
GGGGCGCGCGCCAGGCGGGGCGGGGCGGGGCGAGGGGCGGGGGGGGGCGAGGCG
GAGAGGTGCGGCGGCAGCCAATCAGAGCGGCGCGCTCCGAAAGTTTCCTTTTATGG
CGAGGCGGCGGCGGCGGCGGCCCTATAAAAAGCGAAGCGCGCGGCGGGGGGGAGC
GGGATCAGCCACCGCGGTGGCGGCCTAGAGTCGACGAGGAACTGAAAAACCAGAA
AGTTAACTGGTAAGTTTAGTCTTTTTGTCTTTTATTTCAGGTCCCGGATCCGGTGGTG
GTGCAAATCAAAGAACTGCTCCTCAGTGGATGTTGCCTTTACTTCTAGGCCTGTACG
GAAGTGTTACTTCTGCTCTAAAAGCTGCGGAATTGTACCCGCGGCCGCGTTTAAACC
CTGCAGGTCTAGAAAGCTTATCGATACCGTCGACTAGAGCTCGCTGATCAGCCTCG
ACTGTGCCTTCTAGTTGCCAGCCATCTGTTGTTTGCCCCTCCCCCGTGCCTTGCTTGA
CCCTGGAAGGTGCCACTCCCACTGTCCTTTCCTAATAAAATGAGGAAATTGCATCGC
ATTGTCTGAGTAGGTGTCATTCTATTCTGGGGGGTGGGGTGGGGCAGGACAGCAAG
GGGGAGGATTGGGAAGACAATAGCAGGGTACAAGTAAAGCGGCCCTAGCGTTTCC
GGCGACGGTGCTAGACTCGAGGACGGGGTGAACTACGCCTGAGGATCCGATCTTTT
TCCCTCTGCCAAAAATTATGGGGACATCATGAAGCCCCTTGAGCATCTGACTTCTGG
CTAATAAAGGAAATTTATTTTCATTGCAATAGTGTGTTGGAATTTTTTGTGTCTCTCA
CTCGGAAGCAATTCGTTGATCTGAATTTCGACCACCCATAATACCCATTACCCTGGT
AGATAAGTAGCATGGCGGGTTAATCATTAACTACAAGGAACCCCTAGTGATGGAGT
TGGCCACTCCCTCTCTGCGCGCTCGCTCGCTCACTGAGGCCGGGCGACCAAAGGTCG
CCCGACGCCCGGGCTTTGCCCGGGCGGCCTCAGTGAGCGAGCGAGCGCGCAGCCTT
AATTAACCTAATTCACTGGCCGTCGTTTTACAACGTCGTGACTGGGAAAACCCTGGC
GTTACCCAACTTAATCGCCTTGCAGCACATCCCCCTTTCGCCAGCTGGCGTAATAGC
GAAGAGGCCCGCACCGATCGCCCTTCCCAACAGTTGCGCAGCCTGAATGGCGAATG
GGACGCGCCCTGTAGCGGCGCATTAAGCGCGGCGGGTGTGGTGGTTACGCGCAGCG
TGACCGCTACACTTGCCAGCCCCCTAGCGCCCGCTCCTTTCGCTTTCTTCCCTTCCTT
TCTCGCCACGTTCGCCGGCTTTCCCCGTCAAGCTCTAAATCGGGGGCTCCCTTTAGG
GTTCCGATTTAGTGCTTTACGGCACCTCGACCCCAAAAAACTTGATTAGGGTGATGG
TTCACGTAGTGGGCCATCGCCCTGATAGACGGTTTTTCGCCCTTTGACGTTGGAGTC
CACGTTCTTTAATAGTGGACTCTTGTTCCAAACTGGAACAACACTCAACCCTATCTC
GGTCTATTCTTTTGATTTATAAGGGATTTTGCCGATTTCGGCCTATTGGTTAAAAAAT
GAGCTGATTTAACAAAAATTTAACGCGAATTTTAACAAAATATTAACGCTTACAATT
TAGGTGGCACTTTTCGGGGAAATGTGCGCGGAACCCCTATTTGTTTATTTTTCTAAA
TACATTCAAATATGTATCCGCTCATGAGACAATAACCCTGATAAATGCTTCAATAAT
ATTGAAAAAGGAAGAGTATGAGTATTCAACATTTCCGTGTCGCCCTTATTCCCTTTT
TTGCGGCATTTTGCCTTCCTGTTTTTGCTCACCCAGAAACGCTGGTGAAAGTAAAAG
ATGCTGAAGATCAGTTGGGTGCACGAGTGGGTTACATCGAACTGGATCTCAACAGC
GGTAAGATCCTTGAGAGTTTTCGCCCCGAAGAACGTTTTCCAATGATGAGCACTTTT
AAAGTTCTGCTATGTGGCGCGGTATTATCCCGTATTGACGCCGGGCAAGAGCAACT
CGGTCGCCGCATACACTATTCTCAGAATGACTTGGTTGAGTACTCACCAGTCACAGA
AAAGCATCTTACGGATGGCATGACAGTAAGAGAATTATGCAGTGCTGCCATAACCA
TGAGTGATAACACTGCGGCCAACTTACTTCTGACAACGATCGGAGGACCGAAGGAG
CTAACCGCTTTTTTGCACAACATGGGGGATCATGTAACTCGCCTTGATCGTTGGGAA
CCGGAGCTGAATGAAGCCATACCAAACGACGAGCGTGACACCACGATGGCTGTAGC
AATGGCAACAACGTTGCGCAAACTATTAACTGGCGAACTACTTACTCTAGCTTCCCG
GCAACAATTAATAGACTGGATGGAGGCGGATAAAGTTGCAGGACCACTTCTGCGCT
CGGCCCTTCCGGCTGGCTGGTTTATTGCTGATAAATCTGGAGCCGGTGAGCGTGGGT
CTCGCGGTATCATTGCAGCACTGGGGCCAGATGGTAAGCCCTCCCGTATCGTAGTTA
TCTACACGACGGGGACTCAGGCAACTATGGATGAACGAAATAGACAGATCGCTGA
GATAGGTGCCTCACTGATTAAGCATTGGTAACTGTCAGACCAAGTTTACTCATATAT
ACTTTAGATTGATTTAAAACTTCATTTTTAATTTAAAAGGATCTAGGTGAAGATCCT
TTTTGATAATCTCATGACCAAAATCCCTTAACGTGAGTTTTCGTTCCACTGAGCGTC
AGACCCCGTAGAAAAGATCAAAGGATCTTCTTGAGATCCTTTTTTTCTGCGCGTAAT
CTGCTGCTTGCAAACAAAAAAACCACCGCTACCAGCGGTGGTTTGTTTGCCGGATC
AAGAGCTACCAACTCTTTTTCCGAAGGTAACTGGCTTCAGCAGAGCGCAGATACCA
AATACTGTTCTTCTAGTGTAGCCGTAGTTAGGCCACCACTTCAAGAACTCTGTAGCA
CCGCCTACATACCTCGCTCTGCTAATCCTGTTACCAGTGGCTGCTGCCAGTGGCGAT
AAGTCGTGTCTTACCGGGTTGGACTCAAGACGATAGTTACCGGATAAGGCGCAGCG
GTCGGGCTCAACGGGGGGTTCGTGCACACAGCCCAGCTTGGAGCGAACGACCTACA
CCGAACTGAGATACCTACAGCGTGAGCTATGAGAAAGCGCCACGCTTCCCGAAGGG
AGAAAGGCGGACAGGTATCCGGTAAGCGGCAGGGTCGGAACAGGAGAGCGCACGA
GGGAGCTTCCAGGGGGAAACGCCTGGTATCTTTATAGTCCTGTCGGGTTTCGCCACC
TCTGACTTGAGCGTCGATTTTTGTGATGCTCGTCAGGGGGGCGGAGCCTATGGAAA
AACGCCAGCAACGCGGCCTTTTTACGGTTCCTGGCCTTTTGCTGGCCTTTTGCTCAC
ATGTTCTTTCCTGCGTTATCCCCTGATTCTGTGGATAACCGTATTACCGCCTTTGAGT
GAGCTGATACCGCTCGCCGCAGCCGAACCACCGAGCGCAGCGAGTCATGAGCGA
GGAAGCGGAAGAGCGCCCAATACGCAAACCGCCTCTCCCCGCGGGTTGGCCGATTC
ATTAATGCAGCTGATTCTAACGAGGAAAGCACGTTATACGTGCTCGTCAAAGCAAC
CATAGTACGCGCCCTGTAGCGGCGCATTAAGCGCGGCGGGTGTGGTGGTTACGGGC
AGCGTGACCGCTACACTTGCCAGCGCCCTAGCGCCCGCTCCTTTCGCTTTCTTCCCTT
CCTTTCTCGCCACGTTCGCCGGCTTTCCCCGTCAAGCTCTAAATCGGGGGCTCCCTTT
AGGGTTCCGATTTAGTGCTTTACGGCACCTCGACCCCAAAAAACTTGATTAGGGTGA
TGGTTCACGTAGTGGGCCATGGCCCTGATAGACGGTTTTTCGCCCTTTGACGTTGGA
GTCCACGTTCTTTAATAGTGGACTCTTGTTCCAAACTGGAACAACACTCAACCCTAT
CTCGGTCTATTCTTTTGATTTATAAGGGATTTTGCCGATTTCGGCCTATTGGTTAAAA
AATGAGCTGATTTAACAAAAATTTAACGCGAATTTTAACAAAATATTAACGCTTAC
AATTTAAATATTTGCTTATACAATCTTCCTGTTTTTGGGGCTTTTCTGATTATCAACC
GGGGTACATATGATTGACATGCTAGTTTTACGATTACCGTTCATCGCCCTGCGCGCT
CGCTCGCTCACTGAGGCCCCCCGGGCAAACCCCGGGCGTCGGGCGACCTTTGGTCG
CCCGGCCTCAGTGAGCGAGCGAGCGCGCAGAGAGGGAGTGGAATTCTAAATTCATA
TTTGCATGTCGCTATGTGTTCTGGGAAATCACCATAAACGTGAAATGTCTTTGGATT
TGGGAATCTTATAAGTTCTGTATGAGACCACTCGCCTGGAGGCTTGCTGAAGGCTGT
ATGCTGATGAACATGGAATCCATGCAGGTTTTGGCCACTGACTGACCTGCATGGTCC
ATGTTCATCAGGACACAAGGCCTGTTACTAGCACTCACATGGAACAAATGGCCCTTT
TTTCTAGTGGTAC
> Sequence for CB-anti-Sod1 miR and miRNA resistant Sod1 (SEQ ID NO: 9)
TCAATATTGGCCATTAGCCATATTATTCATTGGTTATATAGCATAAATCAATATTGG
CTATTGGCCATTGCATACGTTGTATCTATATCATAATATGTACATTTATATTGGCTCA
TGTCCAATATGACCGCCATGTTGGCATTGATTATTGACTAGTTATTAATAGTAATCA
ATTACGGGGTCATTAGTTCATAGCCCATATATGGAGTTCCGCGTTACATAACTTACG
GTAAATGGCCCGCCTGGCTGACCGCCCAACGACCCCCGCCCATTGACGTCAATAAT
GACGTATGTTCCCATAGTAACGCCAATAGGGACTTTCCATTGACGTCAATGGGTGG
AGTATTTACGGTAAACTGCCCACTTGGCAGTACATCAAGTGTATCATATGCCAAGTG
CGCCCCCTATTGACGTCAATGACGGTAAATGGCCCGCCTGGCATTATGCCCAGTACA
TGACCTTACGGGACTTTCCTACTTGGCAGTACATCTACGTATTAGTCATCGCTATTA
CCATGGTCGAGGTGAGCCCCACGTTCTGCTTCACTCTCCCCATCTCCCCCCCCTCCCC
ACCCCCAATTTTGTATTTATTTATTTTTTAATTATTTTGTGCAGCGATGGGGGCGGGG
GGGGGGGGGGGGCGCGCGCCAGGCGGGGCGGGGCGGGGCGAGGGGCGGGGCGGG
GCGAGGCGGAGAGGTGCGGCGGCAGCCAATCAGAGCGGCGCGCTCCGAAAGTTTC
CTTTTATGGCGAGGCGGCGGCGGCGGCGGCCCTATAAAAAGCGAAGCGCGCGGCG
GGCGGGACTCGCTGCGACGCTGCCTTCGCCCCGTGCCCCGCTCCGCCGCCGCCTCGC
CCCCCCCCCCCCGGCTCTGACTGACCGCGTTACTCCCACAGGTGAGCGGGCGGGAC
GGCCCTTCTCCTCCGGGCTGTAATTAGCGCTTGGTTTAATGACGGCTTGTTTCTTTTC
TGTGGCTGCGTGAAAGCCTTGAGGGGCTCCGGGAGGGCCCTTTGTGCGGGGGGGAG
CGGCTCGGGGGGTGCGTGCGTGTGTGTGTGCGTGGGGAGCGCCGCGTGCGGCCCGC
GCTGCCCGCCGGCTGTGAGCGCTGCGGGCGCGGCGCGGGGCTTTGTGCGCTCCGCA
GTGTGCGCGAGGGGAGCGCGGCCGGGGGCGGTGCCCCGCGGTGCGGGGGGGGCTG
CGAGGGGAACAAAGGCTGCGTGCGGGGTGTGTGCGTGGGGGGGTGAGCAGGGGGT
GTGGGCGCGGCGGTCGGGCTGTAACCCCCCCCTGCACCCCCCTCCCCGAGTTGCTGA
GCACGGCCCGGCTTCGGGTGCGGGGCTCCGTACGGGGCGTGGCGCGGGGCTCGCCG
TGCCGGGGGGGGGGGGCGGCAGGTGGGGGTGCCGGGGGGGGCGGGGCCGCCTCG
GGCCGGGGAGGGCTCGGGGGAGGGGCGCGGCGGCCCCCGGAGCGCCGGCGGCTGT
CGAGGCGCGGCGAGCCGCAGCCATTGCCTTTTATGGTAATCGTGCGAGAGGGCGCA
GGGACTTCCTTTGTCCCAAATCTGTGCGGAGCCGAAATCTGGGAGGCGCCGCCGCA
CCCCCTCTAGCGGGCGCGGGGCGAAGCGGTGCGGCGCCGGCAGGAAGGAAATGGG
CGGGGAGGGCCTTCGTGCGTCGCCGCGCCGCCGTCCCCTTCTCCCTCTCCAGCCTCG
GGGCTGTCCGCGGGGGGACGGCTGCCTTCGGGGGGGACGGGGCAGGGCGGGGTTC
GGCTTCTGGCGTGTGACCGGCGGCTCTAGCCGGCGACCGGTATGCATCCTGGAGGC
TTGCTGAAGGCTGTATGCTGATGAACATGGAATCCATGCAGGTTTTGGCCACTGACT
GACCTGCATGGTCCATGTTCATCAGGACACAAGGCCTGTTACTAGCACTCACATGG
AACAAATGGCCCCTAGCTCGCGATGCATCTAGAGCCTCTGCTAACCATGTTCATGCC
TTCTTCTTTTTCCTACAGCTCCTGGGCAACGTGCTGGTTATTGTGCTGTCTCATCATT
TTGGCAAAGAATTGCTCGAAGATCTAGGGAATTCGATATCAAGCTTGGGGATTTTCA
GGCACCACCACTGACCTGGGACAGTGTTAACGACACGATCCAATGGCGACGAAGGC
CGTGTGCGTGCTGAAGGGCGACGGCCCAGTGCAGGGCATCATCAATTTCGAGCAGA
AGGAAAGTAATGGACCAGTGAAGGTGTGGGGAAGCATTAAAGGACTGACTGAAGG
CCTGCACGGCTTTCACGTCCACGAGTTTGGAGATAATACAGCAGGCTGTACCAGT
GCAGGTCCTCACTTTAATCCTCTATCCAGAAAACACGGTGGGCCAAAGGATGAAGA
GAGGCATGTTGGAGACTTGGGCAATGTGACTGCTGACAAAGATGGTGTGGCCGATG
TGTCTATTGAAGATTCTGTGATCTCACTCTCAGGAGACCATTGCATCATTGGCCGCA
CACTGGTGGTCCATGAAAAAGCAGATGACTTGGGCAAAGGTGGAAATGAAGAAAG
TACAAAGACAGGAAACGCTGGAAGTCGTTTGGCTTGTGGTGTAATTGGGATCGCCC
AATAAACATTGCCTTGGATGTAGTCTGAGGCCCCTTAACTCATCTGTTATCCTGCTA
GCTGTAGAAATGTATCCTGATAAACATTAAACACTGTAATCTTAAAAGTGTAATTGT
GTGACTTTTTCAGAGTTGCTTTAAAGTACCTGTAGTGAGAAACTGATTTATGATCAC
TTGGAAGATTTGTATAGTTTTATAAAACTCAGTTAAAATGTCTGTTTCAAGGCCGCT
TCGAGCAGACATGATAAGATACATTGATGAGTTTGGACAAACCACAACTAGAATGC
AGTGAAAAAAATGCTTTATTTGTGAAATTTGTGATGCTATTGCTTTATTTGTAACCA
TTATAAGCTGCAATAAACAAGTTAACAACAACAATTGCATTCATTTTATGTTTCAGG
TTCAGGGGGAGATGTGGGAGGTTTTTTAAAGCAAGTAAAACCTCTACAAATGTGGT
AAAATCGA
> Sequence for bicistronic H1-SOD1-miR-CB-SOD1 (SEQ ID NO: 10); miR Resistant SOD1
target is in bold: SOD1 coding sequence in lowercase
AATTCTAAATTCATATTTGCATGTCGCTATGTGTTCTGGGAAATCACCATAAACGTG
AAATGTCTTTGGATTTGGGAATCTTATAAGTTCTGTATGAGACCACTCGCCTGGAGG
CTTGCTGAAGGCTGTATGCTGATGAACATGGAATCCATGCAGGTTTTGGCCACTGAC
TGACCTGCATGGTCCATGTTCATCAGGACACAAGGCCTGTTACTAGCACTCACATGG
AACAAATGGCCCTTTTTTCTAGTGGTACGTCGTTACATAACTTACGGTAAATGGCCC
GCCTGGCTGACCGCCCAACGACCCCCGCCCATTGACGTCAATAATGACGTATGTTCC
CATAGTAACGCCAATAGGGACTTTCCATTGACGTCAATGGGTGGAGTATTTACGGT
AAACTGCCCACTTGGCAGTACATCAAGTGTATCATATGCCAAGTACGCCCCCTATTG
ACGTCAATGACGGTAAATGGCCCGCCTGGCATTATGCCCAGTACATGACCTTATGG
GACTTTCCTACTTGGCAGTACATCTACTCGAGGCCACGTTCTGCTTCACTCTCCCCAT
CTCCCCCCCCTCCCCACCCCCAATTTTGTATTTATTTATTTTTTAATTATTTTGTGCAG
CGATGGGGGCGGGGGGGGGGGGGGGCGCGCGCCAGGCGGGGCGGGGCGGGGGCG
AGGGGCGGGGCGGGGCGAGGCGGAGAGGTGCGGCGGGCAGCCAATCAGAGCGGCGC
GCTCCGAAAGTTTCCTTTTATGGCGAGGCGGCGGCGGCGGCGGCCCTATAAAAAGC
GAAGCGCGCGGCGGGCGGGGAGCGGGATCAGCCACCGCGGTGGCGGCCTAGAGTCG
ACGAGGAACTGAAAAACCAGAAAGTTAACTGGTAAGTTTAGTCTTTTTGTCTTTTAT
TTCAGGTCCCGGATCCGGTGGTGGTGCAAATCAAAGAACTGCTCCTCAGTGGATGTT
GCCTTTACTTCTAGGCCTGTACGGAAGTGTTACTTCTGCTCTAAAAGCTGCGGAATT
GTACCCGCGGCCGATCCAatggcgacgaaggccgtgtgcgtgctgaagggcgacggcccagtgcagggcatcatcaat
ttcgagcagaaggaaagtaatggaccagtgaaggtgtggggaagcattaaaggactgactgaaggcctgcacggctttcacgtccacg
agtttggagataatacagcaggctgtaccagtgcaggtcctcactttaatcctctatccagaaaacacggtgggccaaaggatgaagagag
gcatgttggagacttgggcaatgtgactgctgacaaagatggtgtggccgatgtgtctattgaagattctgtgatctcactctcaggagaccat
tgcatcattggccgcacactggtggtccatgaaaaagcagatgacttgggcaaaggtggaaatgaagaaagtacaaagacaggaaacgc
tggaagtcgtttggcttgtggtgtaattgggatcgcccaataaacattcccttggatgtagtctgaggccccttaactcatctgttatcctgctag
ctgtagaaatgtatcctgataaacattaaacactgtaatcttaaaagtgtaattgtgtgactttttcagagttgctttaaagtacctgtagtgagaa
actgatttatgatcacttggaagatttgtatagttttataaaactcagttaaaatgtctgtttcaaCAGACATGATAAGATACAT
TGATGAGTTTGGACAAACCACAACTAGAATGCAGTGAAAAAAATGCTTTATTTGTG
AAATTTGTGATGCTATTGCTTTATTTGTAACCATTATAAGCTGCAATAAACAAGTTA
ACAACAACAATTGCATTCATTTTATGTTTCAGGTTCAGGGGGAGATGTGGGAGGTTT
TTTAAAGCAAGTAAAACCTCTACAAATGTGGTAAAATCGATAAGGATCT
> Sequence for CB-miR -CB-SOD1 (SEQ ID NO: 11); miR Resistant SOD1 target is in bold;
SOD1 coding sequence in lowercase
TCAATATTGGCCATTAGCCATATTATTCATTGGTTATATAGCATAAATCAATATTGG
CTATTGGCCATTGCATACGTTGTATCTATATCATAATATGTACATTTATATTGGCTCA
TGTCCAATATGACCGCCATGTTGGCATTGATTATTGACTAGTTATTAATAGTAATCA
ATTACGGGGTCATTAGTTCATAGCCCATATATGGAGTTCCGCGTTACATAACTTACG
GTAAATGGCCCGCCTGGCTGACCGCCCAACGACCCCCGCCCATTGACGTCAATAAT
GACGTATGTTGCCATAGTAACGCCAATAGGGACTTTCCATTGACGTCAATGGGTGG
AGTATTTACGGTAAACTGCCCACTTGGCAGTACATCAAGTGTATCATATGCCAAGTC
CGCCCCCTATTGACGTCAATGACGGTAAATGGCCCGCCTGGCATTATGCCCAGTACA
TGACCTTACGGGACTTTCCTACTTGGCAGTACATCTACGTATTAGTCATCGCTATTA
CCATGGTCGAGGTGAGCCCCACGTTCTGCTTCACTCTCCCCATCTCCCCCCCCTCCCC
ACCCCCAATTTTGTATTTATTTATTTTTTAATTATTTTGTGCAGCGATGGGGGCGGGG
GGGGGGGGGGGGCGCGCGCCAGGCGGGGCGGGGCGGGGCGAGGGGCGGGGGCGGG
GCGAGGCGGAGAGGTGCGGCGGCAGCCAATCAGAGCGGCGCGCTCCGAAAGTTTC
CTTTTATGGCGAGGCGGCGGCGGCGGCGGCCCTATAAAAAGCGAAGCGCGCGGCG
GGCGGGAGTCGCTGCGACGCTGCCTTCGCCCCGTGCCCCGCTCCGCCGCCGCCTCGC
GCCGCCCGCCCCGGCTCTGACTGACCGCGTTACTCCCACAGGTGAGCGGGCGGGAC
GCCCCTTCTCCTCCGGGCTGTAATTAGCGCTTGGTTTAATGACGGCTTGTTTCTTTTC
TGTGGCTGCGTGAAAGCCTTGAGGGGCTCCGGGAGGGCCCTTTGTGCGGGGGGGAG
CGGCTCGGGGGGTGCGTGCGTGTGTGTGTGCGTGGGGAGCGCCGCGTGCGGCCCGC
GCTGCCCGGCGGCTGTGAGCGCTGCGGGCGCGGCGCGGGGCTTTGTGCGCTCCGCA
GTGTGCGCGAGGGGAGCGCGGCCGGGGGCGGTGCCCCGCGGTGCGGGGGGGGCTG
CGAGGGGAACAAAGGCTGCGTGCGGGGTGTGTGCGTGGGGGGGTGAGCAGGGGGT
GTGGGCGCGGCGGTCGGGCTGTAACCCCCCCCTGCACCCCCCTCCCCGAGTTGCTGA
GCACGGCCCGGCTTCGGGTGCGGGGCTCCGTACGGGGCGTGGCGCGGGGCTCGCCG
TGCCGGGCGGGGGGTGGCGGCAGGTGGGGGTGCCGGGCGGGGCGGGGCCGCCTCG
GGCCGGGGAGGGCTCGGGGGAGGGGCGCGGCGGCCCCCGGAGCGCCGGCGGCTGT
CGAGGCGCGGCGAGCCGCAGCCATTGCCTTTTATGGTAATCGTGCGAGAGGGCGCA
GGGACTTCCTTTGTCCCAAATCTGTGCGGAGCCGAAATCTGGGAGGCGCCGCCGCA
CCCCCTCTAGCGGGCGCGGGGCGAAGCGGTGCGGCGCCGGCAGGAAGGAAATGGG
CGGGGAGGGCCTTCGTGCGTCGCCGCGCCGCCGTCCCCTTCTCCCTCTCCAGCCTCG
GGGCTGTCCGCGGGGGGACGGCTGCCTTCGGGGGGGACGGGGCAGGGCGGGGGTTC
GGCTTCTGGCGTGTGACCGGCGGCTCTAGCCGGCGACCGGTATGCATCCTGGAGGC
TTGCTGAAGGCTGTATGCTGATGAACATGGAATCCATGCAGGTTTTGGCCACTGACT
GACCTGCATGGTCCATGTTCATCAGGACACAAGGCCTGTTACTAGCACTCACATGG
AACAAATGGCCCCTAGCTCGCGATGCATCTAGAGGCTCTGCTAACCATGTTCATGCC
TTCTTCTTTTTCCTACAGCTCCTGGGCAACGTGCTGGTTATTGTGCTGTCTCATCATT
TTGGCAAAGAATTGCTCGAAGATCTAGGGAATTCGATATCAAGCTTGGGGATTTTCA
GGCACCACCACTGACCTGGGACAGTGTTAACGACACGATCCAatggcgacgaaggccgtgtgcg
tgctgaagggcgacggcccagtgcagggcatcatcaatttcgagcagaaggaaagtaatggaccagtgaaggtgtggggaagcattaaa
ggactgactgaaggcctgcacggctttcacgtccacgagtttggagataatacagcaggctgtaccagtgcaggtcctcactttaatcctct
atccagaaaacacggtgggccaaaggatgaagagaggcatgttggagacttgggcaatgtgactgctgacaaagatggtgtggccgatg
tgtctattgaagattctgtgatctcactctcaggagaccattgcatcattggccgcacactggtggtccatgaaaaagcagatgacttgggca
aaggtggaaatgaagaaagtacaaagacaggaaacgctggaagtcgtttggcttgtggtgtaattgggatcgcccaataaacattcccttg
gatgtagtctgaggccccttaactcatctgttatcctgctagctgtagaaatgtatcctgataaacattaaacactgtaatcttaaaagtgtaattg
tgtgacttttcagagttgctttaaagtacctgtagtgagaaactgatttatgatcacttggaagatttgtatagttttataaaactcagttaaaatgt
ctgtttcasGGCCGCTTCGAGCAGACATGATAAGATACATTGATGAGTTTGGACAAACCA
CAACTAGAATGCAGTGAAAAAAATGCTTTATTTGTGAAATTTGTGATGCTATTGCTT
TATTTGTAACCATTATAAGCTGCAATAAACAAGTTAACAACAACAATTGCATTCATT
TTATGTTTCAGGTTCAGGGGGAGATGTGGGAGGTTTTTTAAAGCAAGTAAAACCTCT
ACAAATGTGGTAAAATCGA
> Sequence for self-complementary H1-SOD1-miR-CB-SOD1 (w/ 3′ UTR)(SEQ ID NO: 12);
AAV ITRs in bold
CCCTGCGCGCTCGCTCGCTCACTGAGGCCGCCCGGGCAAAGCCCGGGCGTCG
GGCGACCTTTGGTCGCCCGGCCTCAGTGAGCGAGCGAGCGCGCAGAGAGGGA
GTGGAAATTCTAAATTCATATTTGCATGTCGCTATGTGTTCTGGGAAATCACCATAA
ACGTGAAATGTCTTTGGATTTGGGAATCTTATAAGTTCTGTATGAGACCACTCGCCT
GGAGGCTTGCTGAAGGCTGTATGCTGATGAACATGGAATCCATGCAGGTTTTGGCC
ACTGACTGACCTGCATGGTCCATGTTCATCAGGACACAAGGCCTGTTACTAGCACTG
ACATGGAACAAATGGCCCTTTTTTCTAGTGGTACGTCGTTACATAACTTACGGTAAA
TGGCCCGCCTGGCTGACCGCCCAACGACCCCCGCCCATTGACGTCAATAATGACGT
ATGTTCCCATAGTAACGCCAATAGGGACTTTCCATTGACGTCAATGGGTGGAGTATT
TACGGTAAACTGCCCACTTGGCAGTACATCAAGTGTATCATATGCCAAGTACGCCCC
CTATTGACGTCAATGACGGTAAATGGCCCGCCTGGCATTATGCCCAGTACATGACCT
TATGGGACTTTCCTACTTGGCAGTACATCTACTCGAGGCCACGTTCTGCTTCACTCTC
CCCATCTCCCCCCCCTCCCCACCCCCAATTTTGTATTTATTTATTTTTTAATTATTTTG
TGCAGCGATGGGGGCGGGGGGGGGGGGGGGGCGCGCGCCAGGCGGGGCGGGGCG
GGGCGAGGGGCGGGGCGGGGCGAGGCGGAGAGGTGCGGCGGCAGCCAATCAGAG
CGGCGCGCTCCGAAAGTTTCCTTTTATGGCGAGGCGGCGGCGGCGGCGGCCCTATA
AAAAGCGAAGCGCGCGGCGGGCGGGAGCGGGATCAGCCACCGCGGTGGCGGCCTA
GAGTCGACGAGGAACTGAAAAACCAGAAAGTTAACTGGTAAGTTTAGTCTTTTTGT
CTTTTATTTCAGGTCCGGGATCCGGTGGTGGTGCAAATCAAAGAACTGCTCCTCAGT
GGATGTTGCCTTTACTTCTAGGCCTGTACGGAAGTGTTACTTCTGCTCTAAAAGCTG
CGGAATTGTACCCGCGGCCGATCCAatggcgacgaaggccgtgtgcgtgctgaagggcgacggcccagtgcag
ggcatcatcaatttcgagcagaaggaaagtaatggaccagtgaaggtgtggggaagcattaaaggactgactgaaggcctgcacggcttt
cacgtccacgagtttggagataatacagcaggctgtaccagtgcaggtcctcactttaatcctctatccagaaaacacggtgggccaaagg
atgaagagaggcatgttggagacttgggcaatgtgactgctgacaaagatggtgtggccgatgtgtctattgaagattctgtgatctcactct
caggagaccattgcatcattggccgcacactggtggtccatgaaaaagcagatgacttgggcaaaggtggaaatgaagaaagtacaaag
acaggaaacgctggaagtcgtttggcttgtggtgtaattgggatcgcccaataaacattcccttggatgtagtctgaggccccttaactcatct
gttatcctgctagctgtagaaatgtatcctgataaacattaaacactgtaatcttaaaagtgtaattgtgtgactttttcagagttgctttaaagtac
ctgtagtgagaaactgatttatgatcacttggaagatttgtatagttttataaaactcagttaaaatgtctgtttcaaCAGACATGATAA
GATACATTGATGAGTTTGGACAAACCACAACTAGAATGCAGTGAAAAAAATGCTTT
ATTTGTGAAATTTGTGATGCTATTGCTTTATTTGTAACCATTATAAGCTGCAATAAA
CAAGTTAACAACAACAATTGCATTCATTTTATGTTTCAGGTTCAGGGGGAGATGTGG
GAGGTTTTTTAAAGCAAGTAAAACCTCTACAAATGTGGTAAAATCGATAAGAAGGA
ACCCCTAGTGATGGAGTTGGCCACTCCCTCTCTGCGCGCTCGCTCGCTCACTG
AGGCCGGGCGACCAAAGGTCGCCCGACGCCCGGGCTTTGCCCGGGCGGCCTC
AGTGAGCGAGCGAGCGCGCAGCCT
> Sequence for self-complementary HI-SOD1-miR-CB-SOD1 (w/o 3′ UTR)(SEQ ID NO: 13);
AAV ITRs in bold
CCCTGCGCGCTCGCTCGCTCACTGAGGCCCCCCGGGCAAAGCCCGGGCGTCG
GGCGACCTTTGGTCGCCCGGCCTCAGTGAGCGAGCGAGCGCGCAGAGAGGGA
GTGGAAATTCTAAATTCATATTTGCATGTCGCTATGTGTTCTGGGAAATCACCATAA
ACGTGAAATGTCTTTGGATTTGGGAATCTTATAAGTTCTGTATGAGACCACTCGCCT
GGAGGCTTGCTGAAGGCTGTATGCTGATGAACATGGAATCCATGCAGGTTTTGGCC
ACTGACTGACCTGCATGGTCCATGTTCATCAGGACACAAGGCCTGTTACTAGCACTC
ACATGGAACAAATGGCCCTTTTTTCTAGTGGTACGTCGTTACATAACTTACGGTAAA
TGGCCCGCCTGGCTGACCGCCCAACGACCCCCGCCCATTGACGTCAATAATGACGT
ATGTTCCCATAGTAACGCCAATAGGGACTTTCCATTGACGTCAATGGGTGGAGTATT
TACGGTAAACTGCCCACTTGGCAGTACATCAAGTGTATCATATGCCAAGTACGCCCC
CTATTGACGTCAATGACGGTAAATGGCCCGCCTGGCATTATGCCCAGTACATGACCT
TATGGGACTTTCCTACTTGGCAGTACATCTACTCGAGGCCACGTTCTGCTTCACTCTC
CCCATCTCCCCCCCCTCCCCACCCCCAATTTTGTATTTATTTATTTTTTAATTATTTTG
TGCAGCGATGGGGGCGGGGGGGGGGGGGGGGCGCGCGCCAGGCGGGGCGGGGCG
GGGCGAGGGGCGGGGCGGGGCGAGGCGGAGAGGTGCGGCGGCAGCCAATCAGAG
CGGCGCGCTCCGAAAGTTTCCTTTTATGGCGAGGCGGCGGCGGCGGCGGCCCTATA
AAAAGCGAAGCGCGCGGCGGGCGGGAGCGGGATCAGCCACCGCGGTGGCGGCCTA
GAGTCGACGAGGAACTGAAAAACCAGAAAGTTAACTGGTAAGTTTAGTCTTTTTGT
CTTTTATTTCAGGTCCCGGATCCGGTGGTGGTGCAAATCAAAGAACTGCTCCTCAGT
GGATGTTGCCTTTACTTCTAGGCCTGTACGGAAGTGTTACTTCTGCTCTAAAAGCTG
CGGAATTGTACCCGCGGCCGATCCAatggcgacgaaggccgtgtgcgtgctgaagggcgacggcccagtgcag
ggcatcatcaatttcgagcagaaggaaagtaatggaccagtgaaggtgtggggaagcattaaaggactgactgaaggcctgcacggcttt
cacgtccacgagtttggagataatacagcaggctgtaccagtgcaggtcctcactttaatcctctatccagaaaacacgggggccaaagg
atgaagagaggcatgttggagacttgggcaatgtgactgctgacaaagatggtgtggccgatgtgtctattgaagattctgtgatctcactct
caggagaccattgcatcattggccgcacactggtggtccatgaaaaagcagatgacttgggcaaaggtggaaatgaagaaagtacaaag
acaggaaacgctggaagtcgtttggcttgtggtgtaattgggatcgcccaataaaCAGACATGATAAGATACATTGAT
GAGTTTGGACAAACCACAACTAGAATGCAGTGAAAAAAATGCTTTATTTGTGAAAT
TTGTGATGCTATTGCTTTATTTGTAACCATTATAAGCTGCAATAAACAAGTTAACAA
CAACAATTGCATTCATTTTATGTTTCAGGTTCAGGGGGAGATGTGGGAGGTTTTTTA
AAGCAACTAAAACCTCTACAAATGTGGTAAAATCGATAAGAAGGAACCCCTAGTG
ATGGAGTTGGCCACTCCCTCTCTGCGCGCTCGCTCGCTCACTGAGGCCGGGCG
ACCAAAGGTCGCCCGACGCCCGGGCTTTGCCCGGGCGGCCTCAGTGAGCGAG
CGAGCGCGCAGCCT
> Sequence for single stranded CB-miR-CB-SOD1 (w/ 3′UTR)(SEQ ID NO: 14); AAV ITRs in
bold
GGGGGGGGGGGGGGGGGTTGGCCACTCCCTCTCTGCGCGCTCGCTCGCTCACT
GAGGCCGGGCGACCAAAGGTCGCCCGACGCCCGGGCTTTGCCCGGGCGGCCT
CAGTGAGCGAGCGAGCGCGCAGAGAGGGAGTGGCCAACTCCATCACTAGGGG
TTCCTAGATCTCAATATTGGCCATTAGCCATATTATTCATTGGTTATATAGCATAAA
TCAATATTGGCTATTGGCCATTGCATACGTTGTATCTATATCATAATATGTACATTTA
TATTGGCTCATGTCCAATATGACCGCCATGTTGGCATTGATTATTGACTAGTTATTA
ATAGTAATCAATTACGGGGTCATTAGTTCATAGCCCATATATGGAGTTCCGCGTTAC
ATAACTTACGGTAAATGGCCCGCCTGGCTGACCGCCCAACGACCCCCGCCCATTGA
CGTCAATAATGACGTATGTTCCCATAGTAACGCCAATAGGGACTTTCCATTGACGTC
AATGGGTGGAGTATTTACGGTAAACTGCCCACTTGGCAGTACATCAAGTGTATCAT
ATGCCAAGTCCGCCCCCTATTGACGTCAATGACGGTAAATGGCCCGCCTGGCATTAT
CCCCAGTACATGACCTTACGGGACTTTCCTACTTGGCAGTACATCTACGTATTAGTC
ATCGCTATTACCATGGTCGAGGTGAGCCCCACGTTCTGCTTCACTCTCCCCATCTGC
CCCCCCTCCCCACCCCCAATTTTGTATTTATTTATTTTTTAATTATTTTGTGCAGCGAT
GGGGGCGGGGGGGGGGGGGGGGCGCGCGCCAGGCGGGGCGGGGCGGGGCGAGGG
GCGGGGCGGGGCGAGGCGGAGAGGTGCGGCGGCAGCCAATCAGAGCGGCGCGCTC
CGAAAGTTTCCTTTTATGGCGAGGCGGCGGCGGCGGCGGCCCTATAAAAAGCGAAG
CGCGCGGCGGGCGGGAGTCGCTGCGACGCTGCCTTCGCCCCGTGCCCCGCTCCGCC
GCCGCCTCGCGCCGCCCGCCCCGGCTCTGACTGACCGCGTTACTCCCACAGGTGAGC
GGGCGGGACGGCCCTTCTCCTCCGGGCTGTAATTAGCGCTTGGTTTAATGACGGCTT
GTTTCTTTTCTGTGGCTGCGTGAAAGCCTTGAGGGGCTCCGGGAGGGCCCTTTGTGC
GGGGGGGAGCGGCTCGGGGGGTGCGTGCGTGTGTGTGTGCGTGGGGAGCGCCGCGT
GCGGCCCGCGCTGCCCGGCGGCTGTGAGCGCTGCGGGCGCGGCGCGGGGCTTTGTG
CGCTCCGCAGTGTGCGCGAGGGGAGCGCGGCCGGGGGCGGTGCCCCGCGGTGCGG
GGGGGGCTGCGAGGGGAACAAAGGCTGCGTGCGGGGTGTGTGCGTGGGGGGGTGA
GCAGGGGGTGTGGGCGCGGCGGTCGGGCTGTAACCCCCCCCTGCACCCCCCTCCCC
GACTTGCTGAGCACGGCCCGGCTTCGGGTGCGGGGCTCCGTACGGGGCGTGGCGCG
GGGCTCGCCGTGCCGGGCGGGGGGGGCGGCAGGTGGGGGTGCCGGGCGGGGCGG
GGCCGCCTGGGGCCGGGGAGGGCTCGGGGGAGGGGCGCGGCGGCCCCCGGAGCGC
CGGCGGCTGTCGAGGCGCGGCGAGCCGCAGCCATTGCCTTTTATGGTAATCGTGCG
AGAGGGCGCAGGGACTTCCTTTGTCCCAAATCTGTGCGGAGCCGAAATCTGGGAGG
CGCCCCCGCACCCCCTCTAGCGGGCGCGGGGCGAAGCGGTGCGGCGCCGGCAGGA
AGGAAATGGGCGGGGAGGGCCTTCGTGCGTCGCCGCGCCGCCGTCCCCTTCTCCCT
CTCCAGCCTCGGGGCTGTCCGCGGGGGGACGGCTGCCTTCGGGGGGGACGGGGCAG
GGCGGGGTTCGGCTTCTGGCGTGTGACCGGCGGCTCTAGCCGGCGACCGGTATGCA
TCCTGGAGCCTTGCTGAAGGCTGTATGCTGATGAACATGGAATCCATGCAGGTTTTG
GCCACTGACTGACCTGCATGGTCCATGTTCATCAGGACACAAGGCCTGTTACTAGCA
CTCACATGGAACAAATGGCCCCTAGCTCGCGATGCATCTAGAGCCTCTGCTAACCAT
GTTCATGCCTTCTTCTTTTTCCTACAGCTCCTGGGCAACGTGCTGGTTATTGTGCTGT
CTCATCATTTTGGCAAAGAATTCCTCGAAGATCTAGGGAATTCGATATCAAGCTTGG
GGATTTTCAGGCACCACCACTGACCTGGGACAGTGTTAACGACACGATCCAatggcgac
gaaggccgtgtgcgtactgaagggcgacggcccagtgcagggcatcatcaatttcgagcagaaggaaagtaatggaccagtgaaggtg
tggggaagcattaaaggactgactgaaggcctgcacggctttcacgtccacgagtttggagataatacagcaggctgtaccagtgcaggtc
ctcactttaatcctctatccagaaaacacggtgggccaaaggatgaagagaggcatgttggagacttgggcaatgtgactgctgacaaaga
tggtgtggccgatgtgtctattgaagattctgtgatctcactctcaggagaccattgcatcattggccgcacactggtggtccatgaaaaagca
gatgacttgggcaaaggtggaaatgaagaaagtacaaagacaggaaacgctggaagtcgtttggcttgtggtgtaattgggatcgcccaat
aaacattcccttggatgtagtctgaggccccttaactcatctgttatcctgctagctgtagaaatgtatcctgataaacattaaacactgtaatctt
aaaagtgtaattgtgtgactttttcagagttgctttaaagtacctgtagtgagaaactgatttatgatcacttggaagatttgtatagttttataaaac
tcagttaaaatgtctgtttcaaGGCCGCTTCGAGCAGACATGATAAGATACATTGATGAGTTTGGA
CAAACCACAACTAGAATGCAGTGAAAAAAATGCTTTATTTGTGAAATTTGTGATGC
TATTGCTTTATTTGTAACCATTATAAGCTGCAATAAACAAGTTAACAACAACAATTG
CATTCATTTTATGTTTCAGGTTCAGGGGGAGATGTGGGAGGTTTTTTAAAGCAAGTA
AAACCTCTACAAATGTGGTAAAATCGACGATAAGGATCTAGGAACCCCTAGTGAT
GGAGTTGGCCACTCCCTCTCTGCGCGCTCGCTCGCTCACTGAGGCCGCCCGGG
CAAAGCCCGGGCGTCGGGCGACCTTTGGTCGCCCGGCCTCAGTGAGCGAGCG
AGCGCGCAGAGAGGGAGTGGCCAA
> Sequence for single stranded CB-miR-CB-SOD1 (w/ 3′UTR)(SEQ ID NO: 15); AAV ITRs in
bold
GGGGGGGGGGGGGGGGGTTGGCCACTCCCTCTCTGCGCGCTCGCTCGCTCACT
GAGGCCGGGCGACCAAAGGTCGCCCGACGCCCGGGCTTTGCCCGGGGGGCCT
CAGTGAGCGAGCGAGCGCGCAGAGAGGGAGTGGCCAACTCCATCACTAGGGG
TTCCTAGATCTCAATATTGGCCATTAGCCATATTATTCATTGGTTATATAGCATAAA
TCAATATTGGCTATTGGCCATTGCATACGTTGTATCTATATCATAATATGTACATTTA
TATTGGCTCATGTCCAATATGACCGCCATGTTGGCATTGATTATTGACTAGTTATTA
ATAGTAATCAATTACGGGGTCATTAGTTCATAGCCCATATATGGAGTTCCGCGTTAC
ATAACTTACGGTAAATGGCCCGCCTGGCTGACCGCCCAACGACCCCGGCCCATTGA
CGTCAATAATGACGTATGTTCCCATAGTAACGCCAATAGGGACTTTCCATTGACGTC
AATGGGTGGAGTATTTACGGTAAACTGCCCACTTGGCAGTACATCAAGTGTATCAT
ATGCCAAGTCCCCCCCCTATTGACGTCAATGACGGTAAATGGCCCGCCTGGCATTAT
CCCCACTACATGACCTTACGGGACTTTCCTACTTGGCAGTACATCTACGTATTAGTC
ATCGCTATTACCATGGTCGAGGTGAGCCCCACGTTCTGCTTCACTCTCCCCATCTCC
CCCCCCTCCCCACCCCCAATTTTGTATTTATTTATTTTTTAATTATTTTGTGCAGCGAT
GGGGGCGGGGGGGGGGGGGGGGCGCGCGCCAGGCGGGGCGGGGCGGGGCGAGGG
GCGGGGCGGGGCGAGGCGGAGAGGTGCGGCGGCAGCCAATCAGAGCGGCGCGCTC
CGAAAGTTTCCTTTTATGGCGAGGCGGCGGCGGCGGCGGCCCTATAAAAAGCGAAG
CGCGCGGCGGGCGGGAGTCGCTGCGACGCTGCCTTCGCCCCGTGCCCCGCTCCGCC
CCCCCCTCGCGCCCCCCGCCCCGGCTCTGACTGACCGCGTTACTCCCACAGGTGAGC
GGGCGGGACGGCCCTTCTCCTCCGGGCTGTAATTACCGCTTGGTTTAATGACGGCTT
GTTTCTTTTCTGTGGCTGCGTGAAAGCCTTGAGGGGCTCCGGGAGGGCCCTTTGTGC
GGGGGGGAGCGGCTCGGGGGGTGCGTGCGTGTGTGTGTGCGTGGGGAGCGCCGCGT
GCGGCCCGCGCTGCCCGGCGGCTGTGAGCGCTGCGGGCGCGGCGCGGGGCTTTGTG
CGCTCCGCAGTGTGCGCGAGGGGAGCGCGGCCGGGGGCGGTGCCCCGCGGTGCGG
GGGGGGCTGCGAGGGGAACAAAGGCTGCGTGCGGGGTGTGTGCGTGGGGGGGTGA
GCAGGGGGTGTGGGCGCGGCGGTCGGGCTGTAACCCCCCCCTGCACCCCCCTCCCC
GAGTTGCTGAGCACGGCCCGGCTTCGGGTGCGGGGCTCCCTACGGGGCGTGGCGCG
GGGCTCGCCGTGCCGGGCGGGGGGTGGCGGCAGGTGGGGGTGCCGGGCGGGGCGG
GGCCGCCTCGGGCCGGGGAGGGCTCGGGGGAGGGGCGCGGCGGCCCCCGGAGCGC
CGGCGGCTGTCGAGGCGCGGCGAGCCGCAGCCATTGCCTTTTATGGTAATCGTGCG
AGAGGGCGCAGGGACTTCCTTTGTCCCAAATCTGTGCGGAGCCGAAATCTGGGAGG
CGCCGCCGCACCCCCTCTAGCGGGCGCGGGGCGAAGCGGTGCGGCGCCGGCAGGA
AGGAAATGGGCGGGGGAGGGCCTTCGTGCGTCGCCGCGCCGCCGTCCCCTTCTCCCT
CTCCAGCCTCGGGGCTGTCCGCGGGGGGACGGCTGCCTTCGGGGGGGACGGGGCAG
GGCGGGGTTCGGCTTCTGGCGTGTGACCGGCGGCTCTAGCCGGCGACCGGTATGCA
TCCTGGAGGCTTGCTGAAGGCTGTATGCTGATGAACATGGAATCCATGCAGGTTTTG
GCCACTGACTGACCTGCATGGTCCATGTTCATCAGGACACAAGGCCTGTTACTAGCA
CTCACATGGAACAAATGGCCCCTAGCTCGCGATGCATCTAGAGCCTCTGCTAACCAT
GTTCATGCCTTCTTCTTTTTCCTACAGCTCCTGGGCAACGTGCTGGTTATTGTGCTGT
CTCATCATTTTGGCAAAGAATTCCTCGAAGATCTAGGGAATTCGATATCAAGCTTGG
GGATTTTCAGGCACCACCACTGACCTGGGACAGTGTTAACGACACGATCCAatggcgac
gaaggccgtgtgcgtgctgaagggcgacggcccagtgcagggcatcatcaatttcgagcagaaggaaagtaatggaccagtgaaggtg
tggggaagcattaaaggactgactgaaggcctgcacggctttcacgtccacgagtttggagataatacagcaggctgtaccagtgcaggtc
ctcactttaatcctctatccagaaaacacgggggccaaaggatgaagagaggcatgttggagacttgggcaatgtgactgctgacaaaga
tggtgtggccgatgtgtctattgaagattctgtgatctcactctcaggagaccattgcatcattggccgcacactggtggtccatgaaaaagca
gatgacttgggcaaaggtggaaatgaagaaagtacaaagacaggaaacgctggaagtcgtttggcttgtggtgtaattgggatcgcccaat
aaaGAGCAGACATGATAAGATACATTGATGAGTTTGGACAAACCACAACTAGAATGC
AGTGAAAAAAATGCTTTATTTGTGAAATTTGTGATGCTATTGCTTTATTTGTAACCA
TTATAAGCTGCAATAAACAAGTTAACAACAACAATTGCATTCATTTTATGTTTCAGG
TTCAGGGGGAGATGTGGGAGGTTTTTTAAAGCAAGTAAAACCTCTACAAATGTGGT
AAAATCGACGATAAGGATCTAGGAACCCCTAGTGATGGAGTTGGCCACTCCCTC
TCTGCGCGCTCGCTCGCTCACTGAGGCCGCCCGGGCAAAGCCCGGGCGTCGG
GCGACCTTTGGTCGCCCGGCCTCAGTGAGCGAGCGAGCGCGCAGAGAGGGAG
TGGCCAA
> SOD1 Promoter insert sequence (SEQ ID NO: 16)
GTGAGCTGAGATTGCACCACTGCACTCCAGCCTGGTGACAGAGTGAGACTCCATAT
CAAAATAAATACATAAATAAATAAAAACAGTGATTCTTAACTGGGAGTGATTTGGC
AACGTCTGGAATTATTTTTGGTTATCCCAGCCTGGCAGGGAGGGACAGGGTATTACT
GGCATCTAGTGAGTAGGGGCTAGGGATTCTACTGAACATCCTACAGTGTACAGGAC
AGCCTCCACAGCAAAGAACTGTCTGGCCCAAAATGTCCATAGTGCCCACATTGGAT
GCCCTGCATTAGGAAGATATAAATACTGTTAAATATCACAGAGTTAAATTCCTTACC
CCTGTTCTAGCAGAGATGATATTCTTGCGGGGGGAGCATCTTCTTGGCTTCAACACA
TTCTTTTCTCCATGGGAGATGATGCCAGAAGAGGGACAGAACAGGGCCCAGTAAAG
CATGGGGCCTGGGGCCAGGGACCCCCTTGTTCAGGTGTGACGACCATCCTACGAAG
GCACCACCCAGGCATCATTAGACCGTCTCAAAAGAAGAGTAATTCACTGTCCCAAA
GCAGCTCTCTCGTGTCTGTGGGCGGATCCCTTGGCAAGTTTACAATGAACTGAAATC
TGCCGAACTTCCTGGAACCCAAAGAAACTTTAGCCTTGGGCAAAGGCCCTTTGGCC
AGCATTTGCACTGTTTATGCAACCGTTTAGAATATACGAATTATCTGGAGACTACTA
CCAAATACAACAGGCAAAACTGCAAATATGTATACTTCCTAGAGGATGATAAAAAA
ATGTGAATTGTATTTCTCTGATAGAGGATGCATTAGAGTCTGAGGGTCTAAATAGCG
TAAATAATAAATAAGTAAATAAATCGATAGTAGTGTACTCCAAACGAGGCTGGAAT
AGCTTCTATTGTTGTTTCACACTGGACTTCAATTAAGTCTCAGTATTTTGCCATACTC
AATATTAACTACTAGGCTGGACGTGGTGGCTCATGTCTGTAATCCCAGCACTTTGGG
AGGCCGAGGTGGGTAGATGGCTGGCTTGAGCTCAGGAGTTTGAAACCAGCCTGGGC
AACATGGTAAAACCCCATCTGTACCCAAAATACAAAAATCAGCCAGGTGTGGTGGC
ACATGCCTGTGGTCCCAGGTACTTGGGAGGCTGAGGCAGGAGGATGGCTTGAACCC
AGGAGGTGGAGGCTGCAGTGAGCTATGATGGCGCCACTGCACTCCAGCCTGGGTGA
CAGAGCGAGACCCTGTCTCAAAAATCAAACAAACAACCCCCTCGCCCCGGACAAAA
GTAGTTTGCACTATTTTCTCATTTCACAATATGTTTTTGAAATATTTCCCTTGAAAGG
TAAGTCATATTTATCATTCCTGTTGTATGGAGGCATCATAAATTATTTCACCATTCTA
CCCTCCTTGAGTGTTGTGGCCTTTAGGCCAGACAAAAACGCAGGTGATGCCTAGAA
GCCAACTAGTTGCCGTTTGGTTATCTGTAGGGTTGTGGCCTTGCCAAACAGGAAAAA
TATAAAAAGAATACCGAATTCTGCCAACCAAATAAGAAACTCTATACTAAGGACTA
AGAAAATTGCAGGGGAAGAAAAGGTAAGTCCCGGGATTGAGGTGTAGCGACTTTCT
ATACCCTCAGAAAACTAAAAAACAAGACAAAAAAATGAAAACTACAAAAGCATCC
ATCTTGGGGCGTCCCAATTGCTGAGTAACAAATGAGACGCTGTGGCCAAACTCAGT
CATAACTAATGACATTTCTAGACAAAGTGACTTCAGATTTTCAAAGCGTACCCTGTT
TACATCATTTTGCCAATTTCGCGTACTGCAACCGGCGGGCCACGCCCCCGTGAAAAG
AAGGTTGTTTTCTCCACATTTCGGGGTTCTGGACGTTTCCCGGCTGCGGGGCGGGGG
GAGTCTCCGGCGCACGCGGCCCCTTGGCCCCGCCCCCAGTCATTCCCGGCCACTGGC
GACCCGAGGCTGCCGCAGGGGGCGGGCTGAGCGCGTGCGAGGCGATTGGTTTGGG
GCCAGAGTGGGCGAGGCGCGGAGGTCTGGCCTATAAAGTAGTCGCGGAGACGGGG
TGCTGGTTTGCGTCGTAGTCTCCTGCAGCGTCTGGGGTTTCCGTTGCAGTCCTCGGA
ACCAGGACCTCGGCGTGGCCTAGCGAGTT
>Wild-type SOD1 amino acid sequence: NCBI Reference Sequence NP_000445.1 (SEQ ID NO:
17)
MATKAVCVLKGDGPVQGIINFEQKESNGPVKVWGSIKGLTEGLHGFHVHEFGDNTAGC
TSAGPHFNPLSRKHGGPKDEERHVGDLGNVTADKDGVADVSIEDSVISLSGDHCIIGRTL
VVHEKADDLGKGGNEESTKTGNAGSRLACGVIGIAQ

1. An isolated nucleic acid comprising:
(a) a first region that encodes one or more first miRNAs comprising a nucleic acid having sufficient sequence complementary with an endogenous mRNA of a subject to hybridize with and inhibit expression of the endogenous mRNA wherein the endogenous mRNA encode a SOD1 protein; and
(b) a second region encoding an exogenous mRNA that encodes a wild-type SOD1 protein,
wherein the one or more first miRNAs do not comp1ise a nucleic acid having sufficient sequence complementary to hybridize with and inhibit expression of the exogenous mRNA.

2. The isolated nucleic acid of claim 1, wherein the exogenous mRNA lacks a 5′ untranslated region (5′ UTR) lacks a 3′ untranslated region (3′ UTR), or lacks both a 5′ UTR and a 3′UTR.

3. The isolated nucleic acid of claim 1, wherein the exogenous mRNA encoding the SOD1 protein has one or more silent base pair mutations relative to the endogenous mRNA, optionally wherein the exogenous mRNA comprises a nucleic acid sequence that is at least 95% identical to the endogenous mRNA.

4. The isolated nucleic acid of claim 1, wherein the wild-type SOD1 protein is encoded by a sequence comprising the sequence set forth in SEQ ID NO: 7 (Hardened SOD1).

5. The isolated nucleic acid of claim 1, wherein the one or more first miRNAs targets an untranslated region (e.g. 5′ UTR or 3′UTR) of the nucleic acid encoding the endogenous mRNA.

6. The isolated nucleic acid of claim 1, wherein the one or more first miRNAs targets a coding sequence of the nucleic acid encoding the endogenous mRNA.

7. The isolated nucleic acid of claim 6, wherein the one or more first miRNAs hybridizes to a nucleic acid comprising 5, 6, 7, 8, 9, 10, 11, 12, 13, 14, 15, 16, 17, 18, 19, 20 or 21 consecutive nucleotides of a RNA encoded by a sequence as set forth in SEQ ID NO: 2.

8. The isolated nucleic acid of claim 6, wherein the one or more first miRNAs is encoded by 5, 6, 7. 8, 9, 10, 11, 12, 13, 14, 15, 16 17, 18, 19, 20 or 21 consecutive nucleotides of a sequence comprising the sequence as set forth in SEQ ID NO: 3 and/or 4.

9. The isolated nucleic acid of claim 8, wherein the one or more first miRNAs further comprise flanking regions of miR-155 or miR-30.

10. The isolated nucleic acid of claim 1, further comprising a first promoter.

11. The isolated nucleic acid of claim 10, wherein the first promoter is operably linked to the first region.

12. The isolated nucleic acid of claim 10, wherein the first promoter is a RNA polymerase III (pol III) promoter, optionally wherein the pol III promoter is an H1 promoter or a U6 promoter.

13. The isolated nucleic acid of claim 10, wherein the first promoter is a RNA polymerase II (pol II) promoter, optionally wherein the pol II promoter is a chicken beta actin (CBA) promoter, or an endogenous SOD1 promoter (e.g., SEQ ID NO: 16).

14. The isolated nucleic acid of claim 10, further comprising a second promoter, wherein the second promoter is operably linked to the second region.

15. The isolated nucleic acid of claim 14, wherein the second promoter us a pol II promoter, optionally wherein the pol II promoter is a chicken beta actin (CBA) promoter, or an endogenous SOD1 promoter.

16. The isolated nucleic acid of claim 1, further comprising an enhancer sequence, optionally wherein the enhancer is a cytomegalovirus (CMV) enhancer.

17. The isolated nucleic acid of claim 1, wherein the first region is positioned within an untranslated region (e.g., UTR) of the second region.

18. The isolated nucleic acid of claim 17, wherein the first region is positioned within an intron of the isolated nucleic acid.

19. The isolated nucleic acid of claim 1, wherein the first region is positioned 5′ with respect to the second region.

20. The isolated nucleic acid of claim 1, further comprising at least one adeno-associated virus (AA V) inverted terminal repeat (ITR).

21-30. (canceled)


SEQUENCE LISTING (17 sequences)

SEQ ID NO: 1 - DNA, 465 bp, Homo sapiens
  source 1..465: mol_type = genomic DNA
atggcgacgaaggccgtgtgcgtgctgaagggcgacggcccagtgcagggcatcatcaatttcgagcagaaggaaagtaatggaccagtgaaggtgtggggaagcattaaaggactgactgaaggcctgcatggattccatgttcatgagtttggagataatacagcaggctgtaccagtgcaggtcctcactttaatcctctatccagaaaacacggtgggccaaaggatgaagagaggcatgttggagacttgggcaatgtgactgctgacaaagatggtgtggccgatgtgtctattgaagattctgtgatctcactctcaggagaccattgcatcattggccgcacactggtggtccatgaaaaagcagatgacttgggcaaaggtggaaatgaagaaagtacaaagacaggaaacgctggaagtcgtttggcttgtggtgtaattgggatcgcccaataa

SEQ ID NO: 2 - DNA, 21 bp, synthetic construct
  misc_feature 1..21: note = Synthetic Polynucleotide
  source 1..21: mol_type = other DNA
ctgcatggattccatgttcat

SEQ ID NO: 3 - DNA, 21 bp, synthetic construct
  misc_feature 1..21: note = Synthetic Polynucleotide
  source 1..21: mol_type = other DNA
gacgtacctaaggtacaagta

SEQ ID NO: 4 - DNA, 21 bp, synthetic construct
  misc_feature 1..21: note = Synthetic Polynucleotide
  source 1..21: mol_type = other DNA
ctgcatggattccatgttcat

SEQ ID NO: 5 - DNA, 64 bp, synthetic construct
  misc_feature 1..64: note = Synthetic Polynucleotide
  source 1..64: mol_type = other DNA
tgctgatgaacatggaatccatgcaggttttggccactgactgacctgcatggtccatgttcat

SEQ ID NO: 6 - DNA, 64 bp, synthetic construct
  misc_feature 1..64: note = Synthetic Polynucleotide
  source 1..64: mol_type = other DNA
atgaacatggaccatgcaggtcagtcagtggccaaaacctgcatggattccatgttcatcagca

SEQ ID NO: 7 - DNA, 465 bp, synthetic construct
  misc_feature 1..465: note = Synthetic Polynucleotide
  source 1..465: mol_type = other DNA
atggcgacgaaggccgtgtgcgtgctgaagggcgacggcccagtgcagggcatcatcaatttcgagcagaaggaaagtaatggaccagtgaaggtgtggggaagcattaaaggactgactgaaggcctgcacggctttcacgtccacgagtttggagataatacagcaggctgtaccagtgcaggtcctcactttaatcctctatccagaaaacacggtgggccaaaggatgaagagaggcatgttggagacttgggcaatgtgactgctgacaaagatggtgtggccgatgtgtctattgaagattctgtgatctcactctcaggagaccattgcatcattggccgcacactggtggtccatgaaaaagcagatgacttgggcaaaggtggaaatgaagaaagtacaaagacaggaaacgctggaagtcgtttggcttgtggtgtaattgggatcgcccaataa

SEQ ID NO: 8 - DNA, 5055 bp, synthetic construct
  misc_feature 1..5055: note = Synthetic Polynucleotide
  source 1..5055: mol_type = other DNA
ctctggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgcgtttaaaccctgcaggtctagaaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcagggtacaagtaaagcggccctagcgtttccggcgacggtgctagactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattctaaattcatatttgcatgtcgctatgtgttctgggaaatcaccataaacgtgaaatgtctttggatttgggaatcttataagttctgtatgagaccactcgcctggaggcttgctgaaggctgtatgctgatgaacatggaatccatgcaggttttggccactgactgacctgcatggtccatgttcatcaggacacaaggcctgttactagcactcacatggaacaaatggcccttttttctagtggtac

SEQ ID NO: 9 - DNA, 3048 bp, synthetic construct
  misc_feature 1..3048: note = Synthetic Polynucleotide
  source 1..3048: mol_type = other DNA
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagccggcgaccggtatgcatcctggaggcttgctgaaggctgtatgctgatgaacatggaatccatgcaggttttggccactgactgacctgcatggtccatgttcatcaggacacaaggcctgttactagcactcacatggaacaaatggcccctagctcgcgatgcatctagagcctctgctaaccatgttcatgccttcttctttttcctacagctcctgggcaacgtgctggttattgtgctgtctcatcattttggcaaagaattcctcgaagatctagggaattcgatatcaagcttggggattttcaggcaccaccactgacctgggacagtgttaacgacacgatccaatggcgacgaaggccgtgtgcgtgctgaagggcgacggcccagtgcagggcatcatcaatttcgagcagaaggaaagtaatggaccagtgaaggtgtggggaagcattaaaggactgactgaaggcctgcacggctttcacgtccacgagtttggagataatacagcaggctgtaccagtgcaggtcctcactttaatcctctatccagaaaacacggtgggccaaaggatgaagagaggcatgttggagacttgggcaatgtgactgctgacaaagatggtgtggccgatgtgtctattgaagattctgtgatctcactctcaggagaccattgcatcattggccgcacactggtggtccatgaaaaagcagatgacttgggcaaaggtggaaatgaagaaagtacaaagacaggaaacgctggaagtcgtttggcttgtggtgtaattgggatcgcccaataaacattcccttggatgtagtctgaggccccttaactcatctgttatcctgctagctgtagaaatgtatcctgataaacattaaacactgtaatcttaaaagtgtaattgtgtgactttttcagagttgctttaaagtacctgtagtgagaaactgatttatgatcacttggaagatttgtatagttttataaaactcagttaaaatgtctgtttcaaggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcga

SEQ ID NO: 10 - DNA, 1956 bp, synthetic construct
  misc_feature 1..1956: note = Synthetic Polynucleotide
  source 1..1956: mol_type = other DNA
aattctaaattcatatttgcatgtcgctatgtgttctgggaaatcaccataaacgtgaaatgtctttggatttgggaatcttataagttctgtatgagaccactcgcctggaggcttgctgaaggctgtatgctgatgaacatggaatccatgcaggttttggccactgactgacctgcatggtccatgttcatcaggacacaaggcctgttactagcactcacatggaacaaatggcccttttttctagtggtacgtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaatggcgacgaaggccgtgtgcgtgctgaagggcgacggcccagtgcagggcatcatcaatttcgagcagaaggaaagtaatggaccagtgaaggtgtggggaagcattaaaggactgactgaaggcctgcacggctttcacgtccacgagtttggagataatacagcaggctgtaccagtgcaggtcctcactttaatcctctatccagaaaacacggtgggccaaaggatgaagagaggcatgttggagacttgggcaatgtgactgctgacaaagatggtgtggccgatgtgtctattgaagattctgtgatctcactctcaggagaccattgcatcattggccgcacactggtggtccatgaaaaagcagatgacttgggcaaaggtggaaatgaagaaagtacaaagacaggaaacgctggaagtcgtttggcttgtggtgtaattgggatcgcccaataaacattcccttggatgtagtctgaggccccttaactcatctgttatcctgctagctgtagaaatgtatcctgataaacattaaacactgtaatcttaaaagtgtaattgtgtgactttttcagagttgctttaaagtacctgtagtgagaaactgatttatgatcacttggaagatttgtatagttttataaaactcagttaaaatgtctgtttcaacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatct

SEQ ID NO: 11 - DNA, 3048 bp, synthetic construct; identical to SEQ ID NO: 9
  misc_feature 1..3048: note = Synthetic Polynucleotide
  source 1..3048: mol_type = other DNA

SEQ ID NO: 12 - DNA, 2194 bp, synthetic construct
  misc_feature 1..2194: note = Synthetic Polynucleotide
  source 1..2194: mol_type = other DNA
ccctgcgcgctcgctcgctcactgaggccgcccgggcaaagcccgggcgtcgggcgacctttggtcgcccggcctcagtgagcgagcgagcgcgcagagagggagtggaaattctaaattcatatttgcatgtcgctatgtgttctgggaaatcaccataaacgtgaaatgtctttggatttgggaatcttataagttctgtatgagaccactcgcctggaggcttgctgaaggctgtatgctgatgaacatggaatccatgcaggttttggccactgactgacctgcatggtccatgttcatcaggacacaaggcctgttactagcactcacatggaacaaatggcccttttttctagtggtacgtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaatggcgacgaaggccgtgtgcgtgctgaagggcgacggcccagtgcagggcatcatcaatttcgagcagaaggaaagtaatggaccagtgaaggtgtggggaagcattaaaggactgactgaaggcctgcacggctttcacgtccacgagtttggagataatacagcaggctgtaccagtgcaggtcctcactttaatcctctatccagaaaacacggtgggccaaaggatgaagagaggcatgttggagacttgggcaatgtgactgctgacaaagatggtgtggccgatgtgtctattgaagattctgtgatctcactctcaggagaccattgcatcattggccgcacactggtggtccatgaaaaagcagatgacttgggcaaaggtggaaatgaagaaagtacaaagacaggaaacgctggaagtcgtttggcttgtggtgtaattgggatcgcccaataaacattcccttggatgtagtctgaggccccttaactcatctgttatcctgctagctgtagaaatgtatcctgataaacattaaacactgtaatcttaaaagtgtaattgtgtgactttttcagagttgctttaaagtacctgtagtgagaaactgatttatgatcacttggaagatttgtatagttttataaaactcagttaaaatgtctgtttcaacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataagaaggaacccctagtgatggagttggccactccctctctgcgcgctcgctcgctcactgaggccgggcgaccaaaggtcgcccgacgcccgggctttgcccgggcggcctcagtgagcgagcgagcgcgcagcct

SEQ ID NO: 13 - DNA, 1979 bp, synthetic construct
  misc_feature 1..1979: note = Synthetic Polynucleotide
  source 1..1979: mol_type = other DNA
ccctgcgcgctcgctcgctcactgaggccgcccgggcaaagcccgggcgtcgggcgacctttggtcgcccggcctcagtgagcgagcgagcgcgcagagagggagtggaaattctaaattcatatttgcatgtcgctatgtgttctgggaaatcaccataaacgtgaaatgtctttggatttgggaatcttataagttctgtatgagaccactcgcctggaggcttgctgaaggctgtatgctgatgaacatggaatccatgcaggttttggccactgactgacctgcatggtccatgttcatcaggacacaaggcctgttactagcactcacatggaacaaatggcccttttttctagtggtacgtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaatggcgacgaaggccgtgtgcgtgctgaagggcgacggcccagtgcagggcatcatcaatttcgagcagaaggaaagtaatggaccagtgaaggtgtggggaagcattaaaggactgactgaaggcctgcacggctttcacgtccacgagtttggagataatacagcaggctgtaccagtgcaggtcctcactttaatcctctatccagaaaacacggtgggccaaaggatgaagagaggcatgttggagacttgggcaatgtgactgctgacaaagatggtgtggccgatgtgtctattgaagattctgtgatctcactctcaggagaccattgcatcattggccgcacactggtggtccatgaaaaagcagatgacttgggcaaaggtggaaatgaagaaagtacaaagacaggaaacgctggaagtcgtttggcttgtggtgtaattgggatcgcccaataaacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataagaaggaacccctagtgatggagttggccactccctctctgcgcgctcgctcgctcactgaggccgggcgaccaaaggtcgcccgacgcccgggctttgcccgggcggcctcagtgagcgagcgagcgcgcagcct

SEQ ID NO: 14 - DNA, 3372 bp, synthetic construct
  misc_feature 1..3372: note = Synthetic Polynucleotide
  source 1..3372: mol_type = other DNA
gggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagccggcgaccggtatgcatcctggaggcttgctgaaggctgtatgctgatgaacatggaatccatgcaggttttggccactgactgacctgcatggtccatgttcatcaggacacaaggcctgttactagcactcacatggaacaaatggcccctagctcgcgatgcatctagagcctctgctaaccatgttcatgccttcttctttttcctacagctcctgggcaacgtgctggttattgtgctgtctcatcattttggcaaagaattcctcgaagatctagggaattcgatatcaagcttggggattttcaggcaccaccactgacctgggacagtgttaacgacacgatccaatggcgacgaaggccgtgtgcgtgctgaagggcgacggcccagtgcagggcatcatcaatttcgagcagaaggaaagtaatggaccagtgaaggtgtggggaagcattaaaggactgactgaaggcctgcacggctttcacgtccacgagtttggagataatacagcaggctgtaccagtgcaggtcctcactttaatcctctatccagaaaacacggtgggccaaaggatgaagagaggcatgttggagacttgggcaatgtgactgctgacaaagatggtgtggccgatgtgtctattgaagattctgtgatctcactctcaggagaccattgcatcattggccgcacactggtggtccatgaaaaagcagatgacttgggcaaaggtggaaatgaagaaagtacaaagacaggaaacgctggaagtcgtttggcttgtggtgtaattgggatcgcccaataaacattcccttggatgtagtctgaggccccttaactcatctgttatcctgctagctgtagaaatgtatcctgataaacattaaacactgtaatcttaaaagtgtaattgtgtgactttttcagagttgctttaaagtacctgtagtgagaaactgatttatgatcacttggaagatttgtatagttttataaaactcagttaaaatgtctgtttcaaggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgacgataaggatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 15 - DNA, 3148 bp, synthetic construct
  misc_feature 1..3148: note = Synthetic Polynucleotide
  source 1..3148: mol_type = other DNA
gggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagccggcgaccggtatgcatcctggaggcttgctgaaggctgtatgctgatgaacatggaatccatgcaggttttggccactgactgacctgcatggtccatgttcatcaggacacaaggcctgttactagcactcacatggaacaaatggcccctagctcgcgatgcatctagagcctctgctaaccatgttcatgccttcttctttttcctacagctcctgggcaacgtgctggttattgtgctgtctcatcattttggcaaagaattcctcgaagatctagggaattcgatatcaagcttggggattttcaggcaccaccactgacctgggacagtgttaacgacacgatccaatggcgacgaaggccgtgtgcgtgctgaagggcgacggcccagtgcagggcatcatcaatttcgagcagaaggaaagtaatggaccagtgaaggtgtggggaagcattaaaggactgactgaaggcctgcacggctttcacgtccacgagtttggagataatacagcaggctgtaccagtgcaggtcctcactttaatcctctatccagaaaacacggtgggccaaaggatgaagagaggcatgttggagacttgggcaatgtgactgctgacaaagatggtgtggccgatgtgtctattgaagattctgtgatctcactctcaggagaccattgcatcattggccgcacactggtggtccatgaaaaagcagatgacttgggcaaaggtggaaatgaagaaagtacaaagacaggaaacgctggaagtcgtttggcttgtggtgtaattgggatcgcccaataaagagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgacgataaggatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 16 - DNA, 2173 bp, Homo sapiens
  source 1..2173: mol_type = genomic DNA
gtgagctgagattgcaccactgcactccagcctggtgacagagtgagactccatatcaaaataaatacataaataaataaaaacagtgattcttaactgggagtgatttggcaacgtctggaattatttttggttatcccagcctggcagggagggacagggtattactggcatctagtgagtaggggctagggattctactgaacatcctacagtgtacaggacagcctccacagcaaagaactgtctggcccaaaatgtccatagtgcccacattcgatgccctgcattaggaagatataaatactcttaaatatcacagagttaaattccttacccctgttctagcagagatgatattcttgcggggggagcatcttcttggcttcaacacattcttttctccatgggagatgatgccagaagagggacagaacagggcccagtaaagcatggggcctggggccagggacccccttgttcaggtgtgacgaccatcctacgaaggcaccacccaggcatcattagaccgtctcaaaagaagagtaattcactgtcccaaagcagctctctcgtgtctgtgggcggatcccttggcaagtttacaatgaactgaaatctgccgaacttcctggaacccaaagaaactttagccttgggcaaaggccctttggccagcatttgcactgtttatgcaaccgtttagaatatacgaattatctggagactactaccaaatacaacaggcaaaactgcaaatatgtatacttcctagaggatgataaaaaaatgtgaattgtatttctctgatagaggatgcattagagtctgagggtctaaatagcgtaaataataaataagtaaataaatcgatagtagtgtactccaaacgaggctggaatagcttctattgttgtttcacactggacttcaattaagtctcagtattttgccatactcaatattaagtactaggctggacgtggtggctcatgtctgtaatcccagcactttgggaggccgaggtgggtagatggctggcttgagctcaggagtttgaaaccagcctgggcaacatggtaaaaccccatctgtacccaaaatacaaaaatcagccaggtgtggtggcacatgcctgtggtcccaggtacttgggaggctgaggcaggaggatggcttgaacccaggaggtggaggctgcagtgagctatgatggcgccactgcactccagcctgggtgacagagcgagaccctgtctcaaaaatcaaacaaacaaccccctcgccccggacaaaagtagtttgcactattttctcatttcacaatatgtttttgaaatatttcccttgaaaggtaagtcatatttatcattcctgttgtatggaggcatcataaattatttcaccattctaccctccttgagtgttgtggcctttaggccagacaaaaacgcaggtgatgcctagaagccaactagttgccgtttggttatctgtagggttgtggccttgccaaacaggaaaaatataaaaagaataccgaattctgccaaccaaataagaaactctatactaaggactaagaaaattgcaggggaagaaaaggtaagtcccgggattgaggtgtagcgactttctataccctcagaaaactaaaaaacaagacaaaaaaatgaaaactacaaaagcatccatcttggggcgtcccaattgctgagtaacaaatgagacgctgtggccaaactcagtcataactaatgacatttctagacaaagtgacttcagattttcaaagcgtaccctgtttacatcattttgccaatttcgcgtactgcaaccggcgggccacgcccccgtgaaaagaaggttgttttctccacatttcggggttctggacgtttcccggctgcggggcggggggagtctccggcgcacgcggccccttggccccgcccccagtcattcccggccactcgcgacccgaggctgccgcagggggcgggctgagcgcgtgcgaggcgattggtttggggccagagtgggcgaggcgcggaggtctggcctataaagtagtcgcggagacggggtgctggtttgcgtcgtagtctcctgcagcgtctggggtttccgttgcagtcctcggaaccaggacctcggcgtggcctagcgagtt

SEQ ID NO: 17 - protein, 154 aa, Homo sapiens [sequence omitted]